(19) United States
(12) Patent Application Publication
(10) Pub. No.: US 2015/0051387 A1
(43) Pub. Date: Feb. 19, 2015

(54) WATER SOLUBLE PLATINUM COMPLEXES FOR TUMOR TREATMENT AND PROCESS OF PREPARING SAME

(71) Applicant: GUDUI BIOPHARMA TECHNOLOGY INC., Huayuan Industrial Zone (CN)

(72) Inventors: Yiqiang Wang, Tianjin (CN); Yang Liu, Tianjin (CN)

(73) Assignee: GUDUI BIOPHARMA TECHNOLOGY INC., Huayuan Industrial Zone (CN)

(21) Appl. No.: 14/369,713

(22) PCT Filed: Jun. 22, 2012
(86) PCT No.: PCT/CN2012/077395

§ 371 Date: Jun. 30, 2014

(30) Foreign Application Priority Data
Jun. 24, 2011 (CN) 201110171539.X
Jun. 20, 2012 (CN) 201210206008.4

(51) Int. Cl. C07H 23/00

(52) U.S. Cl. CPC C07H 23/00
USPC 536/17.9; 536/18.2

(57) ABSTRACT

Disclosed are water-soluble platinum complexes for tumor treatment and preparation method, said platinum complexes being shown as formula (I); The present platinum complexes exhibiting superior cytotoxicity and efficacy compare to the clinical drug oxaliplatin, the design strategy of the present platinum complexes is to enhance the solubility and stability favor its clinical use.

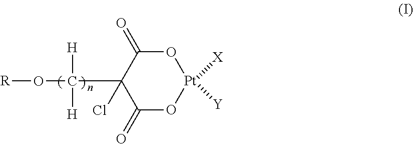

### FIELD OF THE INVENTION

The present invention relates to a water-soluble platinum complex, and especially relates to a water-soluble platinum complex for tumor treatment and a process of preparing the same.

### BACKGROUND OF THE INVENTION

Platinum antitumor drugs are the representative type of the drugs in the field of tumor treatment. They belong to the cell cycle non-specific agents, and the spectrum of antitumor efficacy of them includes sarcoma, malignant epithelial tumor, lymphoma and germ cell tumor. Currently, the world widely used representative platinum anticancer agents in clinic are cisplatin, carboplatin and oxaliplatin.

The fatal weaknesses of platinum anticancer agents are strong toxic side effects and inherent and subsequently acquired resistance. In addition, as these drugs are organometallic compounds, the ubiquitous problem of all listed platinum drugs is the very low water solubility. The following table is the water-soluble data of three above-mentioned clinical drugs:

| Drug | Cisplatin | Carboplatin | Oxaliplatin |
|---|---|---|---|
| Water Solubility | 1.0 | 17.0 | 6.0 |
| (mg/ml) | | | |

The low water solubility brings a lot of adverse effects to the stability of pharmaceutical formulation and clinical applications, for example, it is difficult to successfully formulate them as convenient clinical preparations with an appropriate concentration. Moreover, the low water solubility also directly affects the accumulation and metabolism of the drugs in the body. The metal containing platinum compounds are especially susceptible to the water solubility of the molecule in the aspect of drug excretion, thus the accumulated platinum drugs in the kidney and blood cannot be smoothly excreted from the body and cause strong toxic side effects. As example, the following are drug candidates that represent novel chemical structure but failed to complete clinical trials due to their very low water solubility and drug accumulation based strong side effects and toxicities.

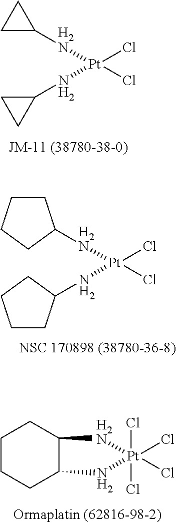

(Reference: Status of platinum drugs in the clinic and in clinical trials, Daiion. Transaciions, 2010, 39, 8113-8127.)

In summary, solve the water solubility problem of the platinum complexes is one of the most important topics in the field of platinum anticancer drug research and development (Galanski, Markus; Keppler, Bernhard K Searching for the Magic Bullet: Anticancer Platinum Drugs Which Can Be Accumulated or Activated in the Tumor Tissue. Anti-Cancer Agents in Medicinal Chemistry, (2007), 7, 55-73). On the one hand, improving the water solubility of platinum drugs is expected to increase the stability of the drugs in vivo, therefore, lower their reactivity with nucleophilic bases in human organs, tissues and cells, and reduce toxic side effects. On the other hand, increase of drug water solubility can help drugs for effective renal excretion, therefore reducing drug accumulation in the body and lowering toxic side effects for kidney and other organs.

### SUMMARY OF THE INVENTION

An object of the invention is to overcome the shortcomings of the existing technology and to substantially change the water solubility problem by providing highly water soluble platinum complexes for tumor treatment.

Another object of the invention is to provide intermediates for preparation of water-soluble platinum complexes for the same purpose.

A further object of the invention is to provide the preparation methods of water-soluble platinum complexes for tumor treatment.

The technical solutions of the present invention are summarized as follows:

The water-soluble platinum complexes for tumor treatment, as of the formula (I):

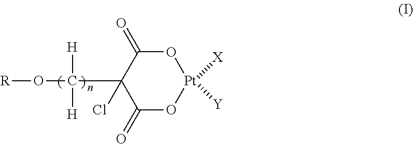

X and Y are ligands, they are the same or independently chosen from NH₃, a C₁-C₈ aliphatic primary amine, a C₃-C₈ cyclic primary amine, an aromatic amine, an aromatic amine containing at least one C₁-C₄ alkyl-substitution group, or a secondary amine with the formula of R₁—NH—R₂, wherein R₁ and R₇, are the same or different represents a C₁-C₈ aliphatic alkyl group, or together forming a C₄-C₈ cyclic alkyl secondary amine, a nitrogen-containing heterocyclic aromatic compound or a nitrogen-containing heterocyclic aromatic compound containing at least one C₁-C₄ alkyl-substitution group, a sulfur-containing heterocyclic aromatic compound or a sulfur-containing heterocyclic non-aromatic compound, or X and Y together as of the formula (VIII):

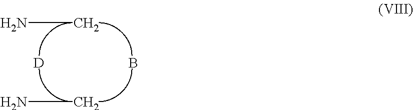

Wherein D is a C₀ or C₁ alkenyl group; B is a C₂-C₈ alkenyl group;

n is an integer from 1 to 6; preferably 1 to 4; the best is 2 or 3;

R is chosen from the following monosaccharides, the 1-position substitution of the monosaccharide are α or β or mixture of both.

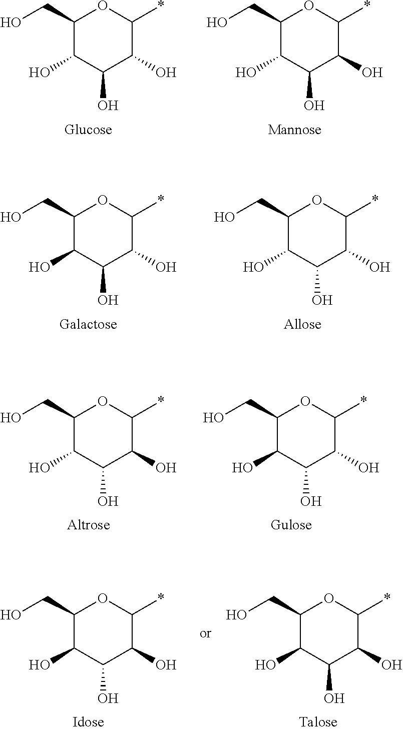

R is preferably chosen from the following monosaccharide, the 1-position substitution of the monosaccharide are α or β or mixture of both.

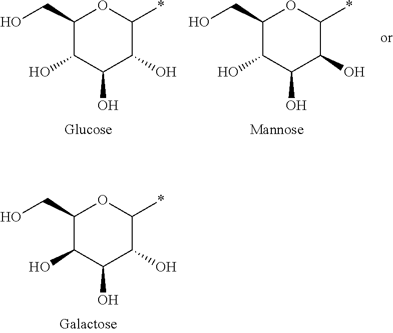 [SMILES: *C1O[C@H](CO)[C@@H](O)[C@H](O)[C@H]1O ; *C1O[C@H](CO)[C@@H](O)[C@H](O)[C@@H]1O ; *C1O[C@H](CO)[C@H](O)[C@H](O)[C@H]1O]

Preferred X and Y are together form a 1,2-cyclohaxanediamine chosen from trans-(1R,2R)-cyclohexanediamine, trans-(1S,2S)-cyclohexanediamine, cis-(1R,2S)-cyclohexanediamine, cis-(1S-2R)-cyclohexanediamine, trans-meso-1,2-cyclohexanediamine, cis-meso-1,2-cyclohexanediamine. The best is trans-(1R,2R)-cyclohexanediamine.

Intermediate used for preparing the provided water-soluble platinum complexes of formula (I) for tumor treatment, shown as the formula (III):

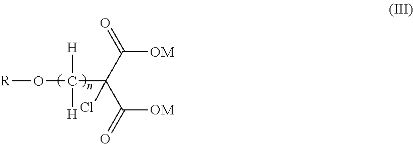

Wherein M chosen hydrogen and metals of elemental periodic table IA, or M together represents a transation metal of elemental periodic table IIA;

n is chosen from 1 to 6, preferably 1 to 4; the best is 2 or 3;

R is chosen from the following monosaccharide, the 1-position substitution of the monosaccharide are α or β or mixture of both.

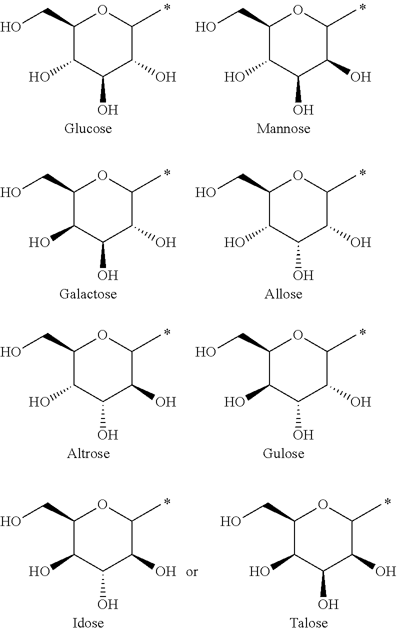

R is preferably chosen from the following monosaccharide, and the 1-position substitution of the monosaccharide are α or β or mixture of both.

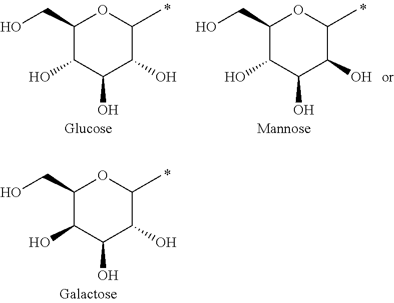

Preparation methods of provided water soluble platinum complexes of formula (I) for tumor treatment, including the following step:

Reacting compound (II) with a water solution of intermediate (III) at a pH range of 7-9, or reacting compound (II) with intermediate (III) in water in the present of inorganic base, where the compound (II) is:

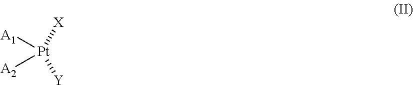

In the formula (II):

X and Y are ligands, they are the same or independently chosen from NH₃, a C₁-C₅₈ aliphatic primary amine, a C₃-C₈ cyclic primary amine, an aromatic amine, an aromatic amine containing at least one C₁-C₄ alkyl-substitution group, or a secondary amine with the formula of R₁—NH—R₂, wherein R₁ and R₂ are the same or different represents a C₁-C₈ aliphatic alkyl group, or together forming a C₄-C₈ cyclic alkyl secondary amine, a nitrogen-containing heterocyclic aromatic compound or a nitrogen-containing heterocyclic aromatic compound containing at least one C₁-C₄ alkyl-substitution group, a sulfur-containing heterocyclic aromatic compound or a sulfur-containing heterocyclic non-aromatic compound, or X and Y together as of the formula (VIII):

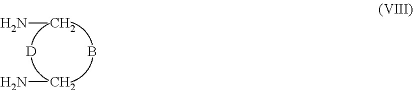

Wherein D is a C₀ or C₁ alkenyl group; B is a C₂-C₈ alkenyl group;

The best example of X and Y of the present invention includes, but is not limited to:

X and Y are each NH₃, isopropylamine, cyclopropylamine, cyclobutylamine, cyclopentylamine, cyclohexylamine; or one of X and Y is NH₃, the other is isopropylamine, cyclopropylamine, cyclobutylamine, cyclopentylamine, cyclohexylamine 2-methylpyridine; 1,2-ethylenediamine, 1,3-diaminopropane, 2-methyl-1,4-diaminobutane, 1,2-cyclobutanediamine, 1,2-cyclopentanediamine, 1,2-cyclohexanediamine, 1,2-cycloheptanediamine, 1,2-cyclooctanediamine, 1-amino-2-aminomethylcyclohexane, 1,1-diaminomethylcyclohexane, 5,5-diaminomethyl-1,3-dioxane, 2-aminomethylpyrrolidine and 2-aminomethylpyridine; when the above ligand compounds contain chiral centers, they can be any one of optical isomers or racemic mixtures;

A₁ and A₂ are the same or different, each represents a hydroxy group, a nitro group, or a perchlorate, or A₁ and A₂ together are sulfate or carbonate;

Intermediate (III) has the following formula:

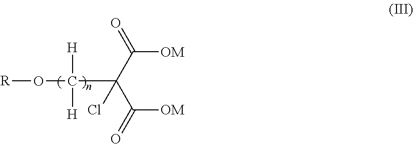

### Wherein:

Each M chosen from hydrogen or metals in Group IA of the elemental periodic table; or M together represents a transition metal in Group IIA of the elemental periodic table; M is preferably chosen from hydrogen, sodium atom, potassium atom, lithium atom or cesium atom; or both M together represents a barium atom;
n is chosen from 1 to 6; preferably 1 to 4; the best is 2 or 3;
R is chosen from the following monosaccharide, the substitution on 1-position of the monosaccharide are α or β or mixture of both.

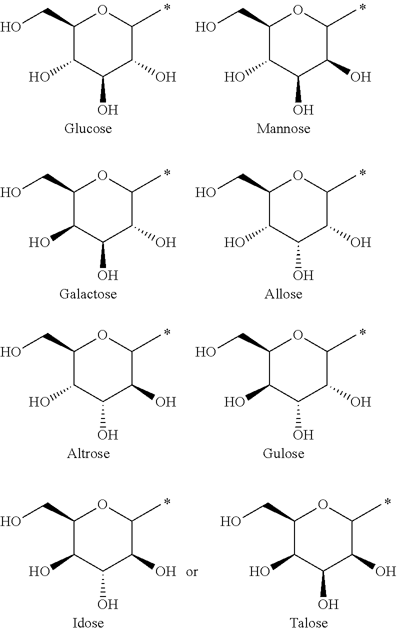

R is preferably chosen from the following monosaccharide, the substitution on 1-position of the monosaccharide are α or β or mixture of both.

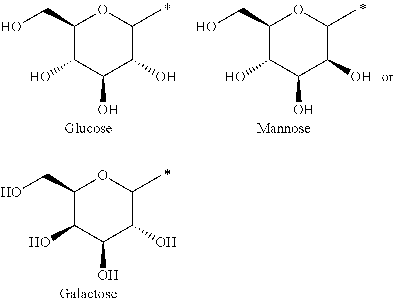

The said inorganic bases are sodium hydroxide, potassium hydroxide, sodium carbonate, sodium bicarbonate, potassium carbonate, lithium hydroxide, barium hydroxide or cesium hydroxide.
Preferred X and Y together are trans-(1R,2R)-cyclohexanediamine, trans-(1S,2S)-cyclohexane diamine, cis-(1R,2S)-cyclohexanediamine, cis-(1S,2R)-cyclohexanediamine, racemic trans-1,2-cyclohexanediamine or racemic cis-1,2-cyclohexanediamine. The best is trans-(1R,2R)-cyclohexanediamine.
Platinum complexes with unprecedented high water-solubility have been achieved in the present invention through introducing water-soluble sugar moiety as substitution group into the structure of traditional malonato-platinum complexes. In addition, as shown in the following formula, like most of the published malonato-platinum compounds, due to the presence of reactive proton at alpha-position of the two carbonyl group in malonato-platinum complexes, the keto-enol tautomerism carry out very easily and resulting in stability problem for such molecules. Especially under the medicinal physiological pH condition, the keto-enol tautomerization will directly lead to decomposition of platinum complexes in vivo (Kresge, A. J. Ingold lecture. Reactive intermediates: Carboxylic acid enols and other unstable species. Chem. Soc. Rev. 1996, 25, 275-280.)

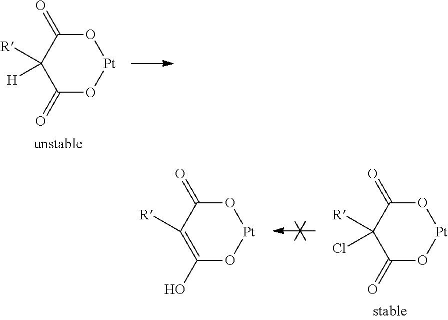

For the above reason, as the present invention, owing to introducing a chlorine atom to the 2-position of the malonato-platinum complexes, the keto-enol tautomerization can be effectively prevented, and furthermore, due to the high concentration of chloride ions in vivo, the stability of the chlorinated platinum complexes can greatly improved when they are used as drugs.
Test result showed that the water solubility of the invented platinum complexes had been increased more than a hundred-fold compare to the clinical drug oxaliplatin; Furthermore, the stability of chlorinated platinum complexes in the current invention had been significantly improved; Thirdly, the animal model efficacy experiments showed that the long-term tumor suppression effect of the chlorinated sugar-containing platinum complexes had been improved and superior to that of oxaliplatin, these results are fully embodying the success and the effects of selective tumor targeting of the water soluble platinum complexes in the present invention. In summary, water-soluble platinum complexes of the present invention can not only solve the poor stability problem of the formulation and the defect of inconvenience in clinical use for the existing platinum drugs, but also improve and enhance the therapeutic efficacy of the existing drugs for tumor therapy.

### BRIEF DESCRIPTION OF THE DRAWINGS

FIG. 1 shows antitumor efficacy-1 of complexes prepared in Example 1

FIG. 2 shows antitumor efficacy-2 of complexes prepared in Example 1

FIG. 3 shows antitumor efficacy-1 of complexes prepared in Example 5

FIG. 4 shows antitumor efficacy-2 of complexes prepared in Example 5

FIG. 5 shows antitumor efficacy-1 of complexes prepared in Example 9

FIG. 6 shows antitumor efficacy-2 of complexes prepared in Example 9

FIG. 7 shows antitumor efficacy-1 of complexes prepared in Example 10

FIG. 8 shows antitumor efficacy-2 of complexes prepared in Example 10

FIG. 9 shows antitumor efficacy of complexes prepared in Example 6, Example 18 and Example 25

### DETAILED DESCRIPTION OF THE INVENTION

The example of the present invention is to enable the skilled artisan in this field better understanding the present invention, but not to limit the present invention in any manner.

As the water-soluble platinum complexes of the present invention for tumor therapy shown as the formula (I), preferred compounds of them can be listed by the following Table 1, but not be limited to the following examples.

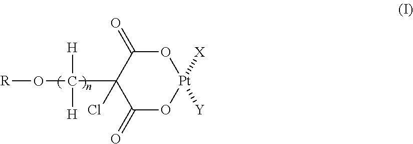

In the formula (I), when R represents a substituent group chosen from D-glucose, D-galactose or D-mannose, n and X, Y are shown in the table 1

TABLE 1

| n | X | Y |
|---|---|---|
| 1-6 | NH₃ | NH₃ |
| 1-6 | isopropylamine | isopropylamine |
| 1-6 | cyclopropylamine | cyclopropylamine |
| 1-6 | cyclobutylamine | cyclobutylamine |
| 1-6 | cyclopentylamine | cyclopentylamine |
| 1-6 | cyclohexylamine | cyclohexylamine |
| 1-6 | NH₃ | cyclobutylamine |
| 1-6 | NH₃ | cyclopentylamine |
| 1-6 | NH₃ | cyclohexylamine |
| 1-6 | NH₃ | 2-methylpyridine |

| | |
|---|---|
| 1-6 | 1,2-ethylenediamine |
| 1-6 | 1,3-propanediamine |
| 1-6 | 1,2-cyclobutanediamine |
| 1-6 | 1,2-cyclopentenediamine |
| 1-6 | 1,2-cyclohexanediamine |
| 1-6 | 1,2-cycloheptanediamine |
| 1-6 | 1,1-diaminomethylcyclohexane |
| 1-6 | 1,2-diaminomethylcyclobutane |
| 1-6 | 2-aminomethylpyridine |

As ligand, 1,2-cyclohexanediamine in Table 1 can be any one of trans-(1R,2R)-cyclohexane diamine, trans-(1S,2S)-cyclohexanediamine, cis-(1R,2S)-cyclohexanediamine, cis-(1S,2R)-cyclohexanediamine, racemic trans-1,2-cyclohexanediamine or racemic cis-1,2-cyclohexanediamine.

The water soluble platinum complexes of the present invention for tumor therapy shown as the formula (I) can be prepared by the following methods.

Method A:

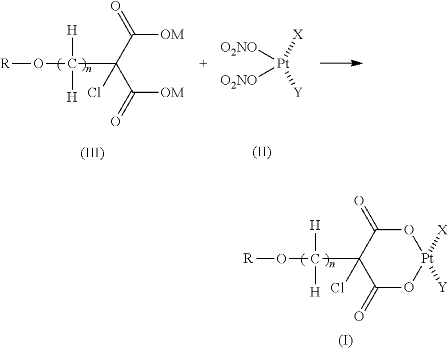

Method B:

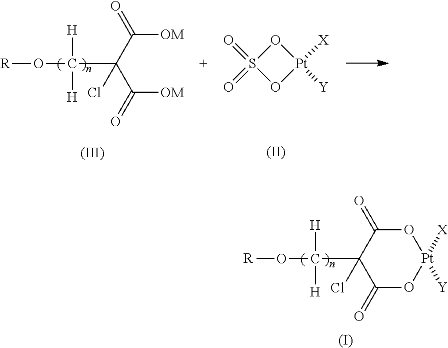

In method A, when M in the formula (III) is a hydrogen atom, the preparation of complexes shown as the formula (I) can be completed by using a suitable inorganic base such as sodium hydroxide, potassium hydroxide, sodium carbonate, sodium bicarbonate, potassium carbonate, lithium hydroxide and cesium hydroxide to maintain the pH of the aqueous reaction solution at a range of 7-9; When M is a metal atom such as a sodium atom, a potassium atom, a barium atom or a cesium atom, the preparation can be carried out smoothly in an aqueous solution, if necessary, a small amount of an aqueous solution of the inorganic base can be used to maintain the pH of the reaction solution at 7-9.

In method B, when M is a hydrogen atom, the preparation of complexes shown as the formula (I) can be completed by the condensation reaction with the platinum sulfate compound shown as the formula (II) with an equivalent amount of barium hydroxide as an inorganic base in aqueous solution. The preparation can also be completed by reacting the barium salt of compound (III) (that is, both M together are a barium atom) with platinum sulfate complexes shown as the formula (II) in an aqueous solution.

The above reaction solvent is preferable to use deionized water; the reaction temperature is generally at room temperature or at 60-90° C. as needed.

The compounds shown as the formula (II) in method A and B can be prepared by reacting corresponding complexes of X, Y coordinated cis-platinum dichloride with silver nitrate or silver sulfate, for example: reacting cis-dichloro-(1,2-diaminocyclohexane) platinum complex with two equivalents of silver nitrate or one equivalent of silver sulfate. The reaction is preferably carried out in an aqueous solution, the deionized water is preferably used, and the reaction temperature is preferable at room temperature.

The thus obtained compound (II) was reacted with the pre-prepared compound (III) in distilled or deionized water as the solvent. 0.5-4 equivalents of compound (II) were needed for each equivalent of the compound (III), the preferred amount is 1 to 2 equivalents. The reaction was completed at pH 7-9, which can be maintained by using a suitable base. The best is an inorganic base, such as sodium hydroxide, potassium hydroxide, barium hydroxide, sodium carbonate, potassium carbonate, sodium bicarbonate. The aqueous solution of these bases with approximate equivalent concentration (1N) was preferably used. The reaction can be carried out within a relatively wide temperature range, for example, 0-100° C., and preferably from room temperature to 90° C., and at the same time with stirring as well. The reaction time varies considerably according to the different target compounds. Depending on the nature of the reactants, the reaction time need generally 1 hour to 30 days, and more often is 10 hours to 15 days.

Many methods can be used to purify the product of formula (I) obtained in the above reaction. For example, the completed reaction mixture can be first filtrated to remove the precipitate that may be generated, and then concentrated by distillation under reduced pressure, and then an organic solvent was added to precipitate out the desired platinum complex of formula (I). An organic solvent which miscible with water is usually selected, such as an alcohol (e.g., methanol, ethanol, propanol, butanol, isopropanol, etc.), or an ether that has a certain miscibility with water (e.g. diethyl ether, methyl tert-butyl ether, THF, ethylene glycol diethyl ether, ethylene glycol dimethyl ether, etc.). Finally the obtained precipitate was collected, for example by filtration, and then the complexes of formula (I) can be achieved. The product obtained above can also be purified and refined by chromatography, etc. for example, by ion exchange resins, or by preparative liquid chromatography. Methanol and water are usually used as fraction of the collected product during separation and purification by liquid chromatography.

In case of glucose, the compound (III) of the present invention can be prepared by each of the following methods: C, D or method E, F.

Method C:

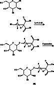

Method D:

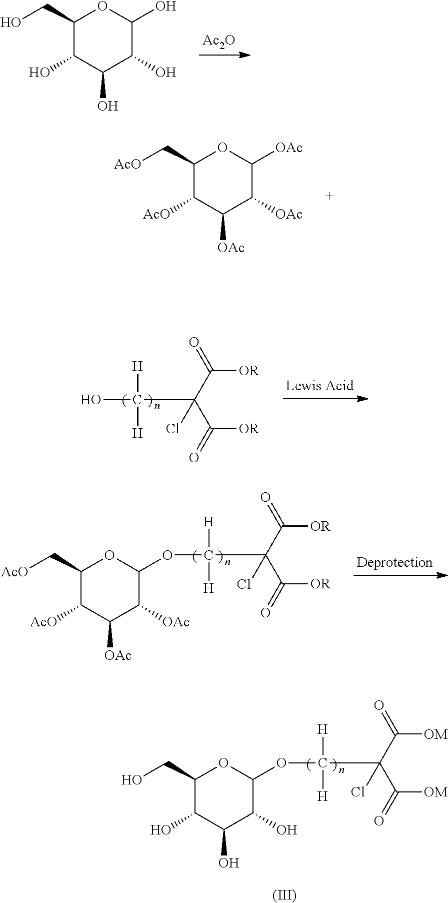

Method E:

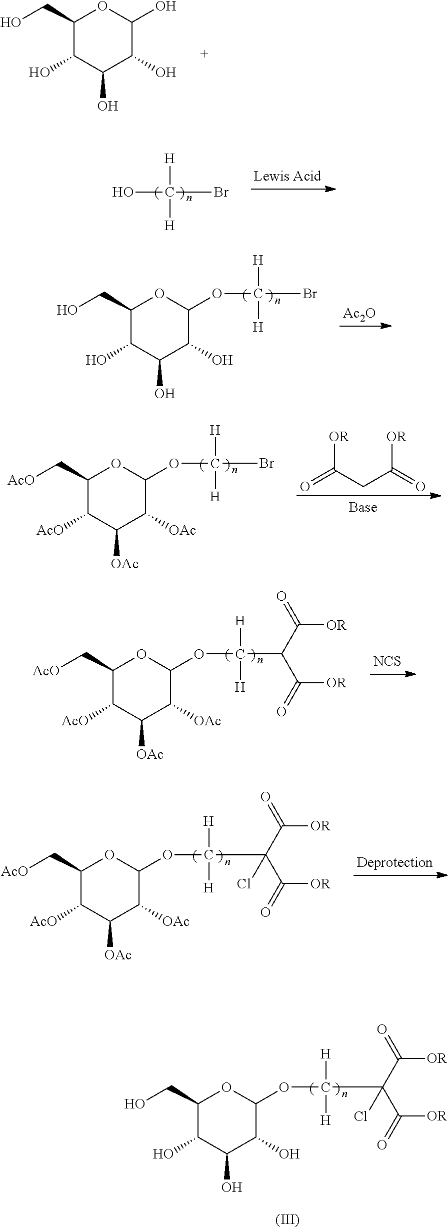

Method F:

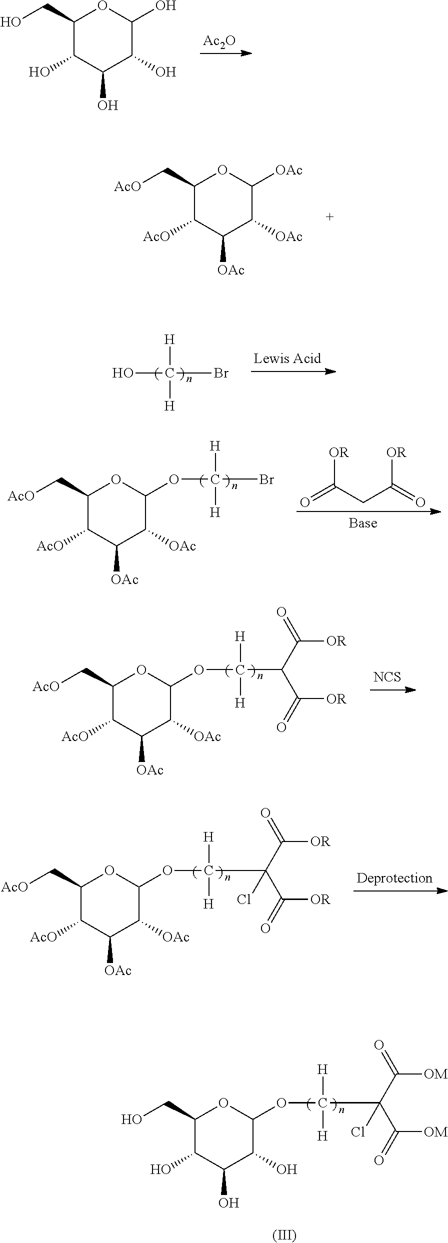

In case of glucose, in method C, 2-chloro substituted malonate derivatives, can be prepared by reacting a halogenated alkyl alcohol with 2-chloromalonate derivatives such as dimethyl chloromalonate, diethyl chloromalonate, dibenzyl chloromalonate and 2-chloromalonic acid cyclic isopropylidene ester and the like according to the general methods known in the literature (e.g. Journal of the American Chemical Society, 131(8), 2786-2787: 2009). Then condensation reaction of the resulting 2-chloro-2-hydroxyalkyl malonate derivatives with D-glucose in the presence of a Lewis acid can produce the corresponding glucoside compounds. 0.1-50 equivalents of 2-chloromalonate derivatives in respect to glucose was used in the condensation reaction, or on the contrary, 0.1-50 equivalents of glucose in respect to 2-chloromalonate compounds was used. The Lewis acid may be chosen from BF₃, SnCl₄, FeCl₃, AlCl₃, hydrochloric acid, p-toluenesulfonic acid, camphorsulfonic acid, etc. The amount of Lewis acid can be 0.1-10 equivalents in respect to glucose. The solvent can be selected from THF, dichloromethane, toluene, ethylene glycol dimethyl ether, ethylene glycol diethyl ether, etc. Any one of the two reactants can also be chose as the solvent. The reaction temperature can be from 0 to 100° C., generally at 60-80° C. The reaction time differs depending on the reactants, generally ranging from 1 hour to 7 days. The resulting products can be refined by a series of purification methods, generally by silica gel column chromatography or by liquid chromatography. The obtained product, after removal of the protecting group of malonic acid, can give the desired compounds shown as the formula (III). The method of deprotection differs depending on the protecting group, for example, benzyl group can be removed by hydrogenation, and diethyl group and isopropylidene group can be deprotected by using an inorganic base with methanol-water, or THF-water as solvent, the ratio of organic solvent to water is generally 1:1-4:1. The inorganic bases can be chose from sodium hydroxide, potassium hydroxide, barium hydroxide and lithium hydroxide, etc. The reaction temperature for the deprotection reaction is usually from room temperature to 60° C., the reaction time generally ranges from 1 to 24 hours. The compound after deprotection can be purified by silica gel column chromatography or ion exchange resin, or by liquid chromatography. If the reaction solvent can be removed directly by distillation, the resulting product will be the corresponding metal carboxylate salt of formula (III).

As shown in Method D, D-glucose can also be firstly converted into the corresponding acetylated glucose, and then react with the 2-chloromalonate derivatives. D-glucose can be acetylated in accordance with the method reported in the literature, for example, the acetylation can be completed in pyridine with acetic anhydride as the acetylating agent at room temperature or at 60° C. for 1-24 hours. Other steps and conditions except the acetylation in Method D, are the same as described in Method C.

In method E and F, halohydrin are firstly coupled with glucose or acetylated glucose in the presence of a Lewis acid, and then the obtained glucoside react with malonate followed by the chlorination, and finally produce the compound (III). Chloro substitution at 2-position of malonate can be accomplished by using the NCS as a representative chlorinating reagent. The reaction is completed in DMF, THF or diethyl ether by treating malonate with one equivalent or excess amount of the base and then the chlorinating reagent. The base may be chosen from sodium hydride, potassium carbonate, sodium carbonate, cesium carbonate, sodium bicarbonate, etc. The equivalent of chlorinating reagent is 1-3 times of the malonate, the reaction temperature is generally from 0° C. to 60° C., preferably at room temperature with stirring. Except the chlorination reaction, all other reaction conditions involved in acetylation of glucose, glycosidation reaction in the presence of Lewis acid, base mediated alkylation reaction at 2-position of the malonate and the final deprotection reaction, are the same as described in method C and D.

The Main Experimental Apparatus:

NMR spectrometer: BRUKER AVANCE III, 400 MHz; Liquid Chromatography for Analysis: Beijing Tong Heng Innovation LC3000 high performance liquid chromatograph, with SPD-10ATvp dual wavelength UV detector, 7725i manual injector, CLASS-VP chromatography workstation; Analytical HPLC Column: DaisoGel, C₁₈, 4.6×250 cm, 5 μm KNAUER Germany; Semi-preparative Liquid Chromatography: Beijing Tong Heng Innovation LC3000 semi-preparative liquid chromatography, SPI001; Semi-preparative Column. DaisoGel 250×20 mm ID, C18, 10 μm; Mass Spectrometer: Agilent 6310 Ion Trap LC/MS; Lyophilizer: FD-lc-50 lyophilizer (Beijing Boyikang Laboratory Instruments Co., Ltd).

### Example 1

### (1) Preparation of 1-O-D-glucoside-2-bromoethane (IV-1)

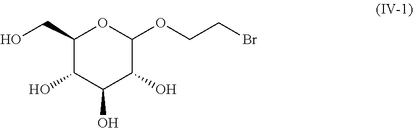 [SMILES: OC[C@H]1OC(OCCBr)[C@H](O)[C@@H](O)[C@@H]1O]

1) To 2-bromoethanol (10 mL) was added glucose (2.7 g) at room temperature, and then cooled to 0° C. The air inside the flask was replaced with nitrogen, then 1 mL of BF₃-Et₂O complex was added dropwise under a nitrogen atmosphere;

2) The reaction solution was stirred at 0° C. for 15 minutes, then slowly warmed to room temperature and stirred for 30 minutes, then heated to 80° C. and stirred for 5 hours; After completion of the reaction, the solvent was evaporated in vacuum and the residue was simply subjected to purification on silica gel chromatography (CH₂Cl₂/CH₃OH: 6/1) to give the crude product (IV-1). Yield: 2.3 g. MS, m/z: 287.23 [M+H]+

### (2) Preparation of 1-O-(2,3,4,6-tetra-acetyl-D-glucoside)-2-bromoethane (V-1)

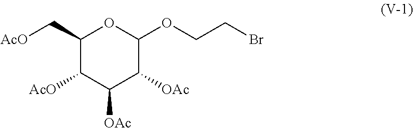 [SMILES: CC(=O)OC[C@H]1OC(OCCBr)[C@H](OC(C)=O)[C@@H](OC(C)=O)[C@@H]1OC(C)=O]

2.3 g of 1-O-D-glucoside-2-bromoethane (IV-1) obtained in the previous step was dissolved in pyridine and acetic anhydride (7 mL: 7 mL) at room temperature, then the reaction mixture was stirred overnight and the reaction was monitored by TLC. After completion of the reaction, ethyl acetate (100 mL) was added, washed with 5% (volume concentration) aqueous hydrochloric acid (2×25 mL), re-extracted with ethyl acetate (2×25 mL), then the organic phase was combined and washed with saturated NH4Cl (aq.) (1×100 mL), water (1×100), saturated NaHCO3 (aq.) (1×100 mL), brine (1×100 mL), dried over anhydrous sodium sulfate and concentrated under reduced pressure to give the pale yellow crude product. The obtained crude product was purified by silica gel column chromatography (PE/EtOAc:3/1) to give the desired product as a colorless oil (V-1). Yield: 2.5 g.

1H NMR (400 MHz, CDCl3): δ5.45 (t, J=9.6 Hz, 1H), 5.15 (d, J=4 Hz, 1H), 5.02 (t, J=9.6 Hz, 1H), 4.80-4.83 (m, 1H), 4.19-4.23 (m, 1H), 4.04-4.15 (m, 2H), 3.92-4.00 (m, 1H), 3.75-3.85 (m, 1H), 3.49 (t, J=6 Hz, 2H), 1.91-2.11 (m, 12H). MS, m/z: 455.15 [M+H]+

### (3) Preparation of 1-O-(2,3,4,6-tetra-acetyyl-D-glucoside)-propane-3,3-diethyl dicarboxylate (VI-1)

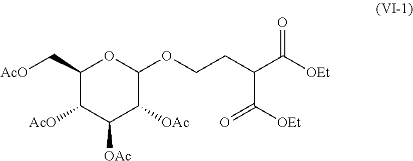 [SMILES: CCOC(=O)C(CCOC1O[C@H](COC(C)=O)[C@@H](OC(C)=O)[C@H](OC(C)=O)[C@H]1OC(C)=O)C(=O)OCC]

2.5 g of 1-O-(2,3,4,6-tetra-acetyl-D-glucoside)-2-bromoethane (V-1) obtained in the previous step was dissolved in dry DMF (5 mL), to which was added potassium carbonate (3 g) followed by diethyl malonate (1.76 g), then the reaction mixture was stirred at room temperature overnight and the reaction was monitored by TLC. After completion of the reaction, ethyl acetate (100 mL) was added, washed with saturated NH4Cl (aq.) (1×50 mL), re-extracted with ethyl acetate (2×25 mL), then the organic phase was combined and washed with saturated NH4Cl (aq.) (1×100 mL), water (1×100 mL), brine (1×100 mL), dried over anhydrous sodium sulfate and concentrated under reduced pressure to give the pale yellow crude product. The obtained crude product was purified by silica gel column chromatography (PE/EtOAc: 3/1) to give the desired product as a colorless oil (VI-1). Yield: 2.6 g.

1H NMR (400 MHz, CDCl3): δ5.42 (t, J=9.6 Hz, 1H), 4.96-5.10 (m, 2H), 4.78-4.90 (m, 1H), 4.03-4.33 (m, 5H), 3.92-4.02 (m, 1H), 3.71-3.87 (m, 1H), 3.71-3.87 (m, 1H), 3.55 (t, J=8 Hz, 1H), 3.40-3.50 (m, 1H), 2.13-2.28 (m, 2H), 1.94-2.14 (m, 12H), 1.15-1.35 (m, 6H). MS, m/z: 535.34 [M+H]+

### (4) Preparation of 1-O-(2,3,4,6-tetra-acetyl-D-glucoside)-propane-3-chloro-3,3-diethyl dicarboxylate (VII-1)

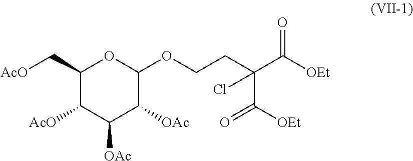 [SMILES: CCOC(=O)C(Cl)(CCOC1O[C@H](COC(C)=O)[C@@H](OC(C)=O)[C@H](OC(C)=O)[C@H]1OC(C)=O)C(=O)OCC]

2.6 g of 1-O-(2,3,4,6-tetra-acetyl-D-glucoside)-propane-3,3-diethyl dicarboxylate was dissolved in dry THF (20 mL), and then cooled to 0° C. The air inside the flask was replaced with nitrogen, and to the above solution was slowly added solid sodium hydride (235 mg, 60% suspension in mineral oil) under a nitrogen atmosphere and stirred for 1 hour after warming to room temperature, then NCS (780 mg) was added and stirred for 2 hours at room temperature, the solvent was removed by rotary evaporation. Ethyl acetate (100 mL) was added, washed with saturated NH4Cl (aq.) (1×50 mL), re-extracted with ethyl acetate (2×25 mL), then the organic phase was combined and washed with saturated NH4Cl (aq.) (1×100 mL), water (1×100 mL), brine (1×100 mL), dried over anhydrous sodium sulfate and concentrated under reduced pressure to give the pale yellow crude product. The obtained crude product was purified by silica gel column chromatography (PE/EtOAc:3/1) to give the desired product as a colorless oil (VII-1). Yield: 2.6 g.

1H NMR (400 MHz, CDCl3): δ5.29 (t, J=9.6 Hz, 1H), 4.90-5.00 (m, 2H), 4.67-4.78 (m, 1H), 4.15-4.35 (m, 5H), 3.97-4.05 (m, 2H), 3.85-3.95 (m, 1H), 3.45-3.55 (m, 1H), 2.48-2.65 (m, 2H), 1.85-2.05 (m, 12H), 1.10-1.30 (m, 6H). MS, m/z: 569.19 [M+H]+

### (5) Preparation of 1-O-D-glucoside-propane-3-chloro-3,3-dicarboxylic acid (III-1)

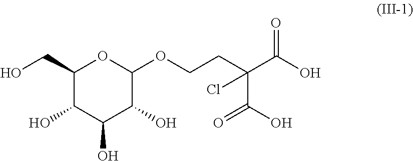 [SMILES: O=C(O)C(Cl)(CCOC1O[C@H](CO)[C@@H](O)[C@H](O)[C@H]1O)C(=O)O]

1) 2.6 g of 1-O-(2,3,4,6-tetra-acetyl-D-glucoside)-propane-3-chloro-3,3-diethyl dicarboxylate was dissolved in methanol (5 mL), to which was then added a solution of sodium hydroxide (1.5 g) dissolved in water (10 mL) at room temperature and then heated at 60° C. for 24 hours. The reaction was monitored by TLC.

2) After completion of the reaction, the methanol was removed by rotary evaporation, then the residue was treated with strong acid cation exchange resin. The aqueous filtrate obtained from filtration of the resin was lyophilized to give a colorless viscous liquid (1.5 g). The crude product was used directly in the next step. MS, m/z: 345.11 [M+H]+

### (6) Preparation of cis-[trans-(1R,2R)-diamino cyclohexane] Pt (II) (1-O-D-glucoside-propane-3-chloro-3,3-dicarboxylate) (I-1)

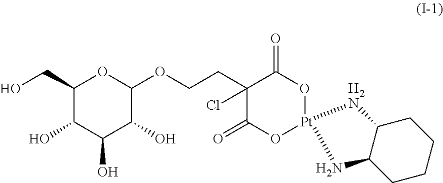

1) 1.5 g of crude 1-O-D-glucoside-propane-3-chloro-3,3-dicarboxylic acid was dissolved in water (15 mL), to which was added a freshly prepared aqueous solution of barium hydroxide to adjust the pH to 7, and then stirred at room temperature for 30 minutes.

2) Under the protection of nitrogen, to the reaction solution of 1) was added a solution of trans-(1R,2R)-diaminocyclohexane platinum sulfate (1.7 g) dissolved in water (2 mL) followed by a freshly prepared aqueous solution of barium hydroxide to re-adjust the pH to 7. The reaction mixture was stirred in the dark at a room temperature overnight.

3) After completion of the reaction, the precipitate was removed by centrifugation, the supernatant was collected and purified by semi-preparative HPLC, and the fraction of the collected product was lyophilized by a lyophilizer to obtain the final product (I-1) as a white solid. Yield: 1.5 g.

1H NMR (400 MHz, D2O): δ5.76 (s, 1H), 5.67 (s, 1H), 5.15 (s, 1H), 4.96 (s, 1H), 4.84 (d, J=3.6 Hz, 0.8H, α-isomer), 4.40 (d, J=7.2 Hz, 0.2H, β-isomer), 3.20-4.00 (m, 10H), 2.20-2.45 (s, 2H), 1.95 (d, J=12 Hz, 2H), 1.48 (d, J=8 Hz, 2H), 1.12-1.30 (s, 2H), 0.95-1.10 (m, 2H). MS, m/z: 652.36 [M+H]+

### Example 2

### (1) Preparation of 1-O-D-glucoside-3-bromopropane (IV-2)

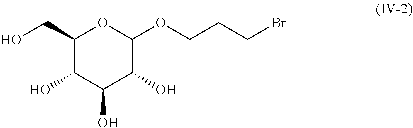 [SMILES: OC[C@H]1OC(OCCCBr)[C@H](O)[C@@H](O)[C@@H]1O]

1) To 3-bromopropanol (8 mL) was added glucose (2.7 g) at room temperature, and then cooled to 0° C., the air inside the flask was replaced with nitrogen, then 0.7 mL of BF3-Et2O complex was added dropwise under a nitrogen atmosphere;

2) The reaction solution was stirred at 0° C. for 15 minutes, then slowly warmed to room temperature and stirred for 30 minutes, then heated to 80° C. and stirred for 5 hours. After completion of the reaction, the solvent was evaporated in vacuum and the residue was simply subjected to purification on silica gel chromatography (CH2Cl2/CH3OH: 6/1) to give the crude product (IV-2). Yield: 2 g. MS, m/z: 301.23 [M+H]+

### (2) Preparation of 1-O-(2,3,4,6-tetra-acetyl-D-glucoside)-3-bromopropane (V-2)

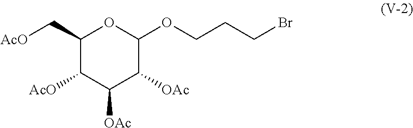 [SMILES: CC(=O)OC[C@H]1OC(OCCCBr)[C@H](OC(C)=O)[C@@H](OC(C)=O)[C@@H]1OC(C)=O]

2 g of 1-O-D-glucoside-3-bromopropane (V-2) obtained in the previous step was dissolved in pyridine and acetic anhydride (6 mL:6 mL) at room temperature, then the reaction mixture was stirred overnight and the reaction was monitored by TLC. After completion of the reaction, ethyl acetate (100 mL) was added, washed with 5% (volume concentration) of aqueous hydrochloric acid (2×25 mL), re-extracted with ethyl acetate (2×25 mL), then the organic phase was combined and washed with saturated NH4Cl (aq.) (1×100 mL), water (1×100 mL), saturated NaHCO3 (aq.) (1×100 mL), brine (1×100 mL), dried over anhydrous sodium sulfate and concentrated under reduced pressure to give the pale yellow crude product. The obtained crude product was purified by silica gel column chromatography (PE/EtOAc:3/1) to give the desired product as a colorless oil (V-2). Yield: 2.1 g.

1H NMR (400 MHz, CDCl3): δ5.47 (t, J=9.6 Hz, 1H,), 5.00-5.15 (m, 2H), 4.85-4.95 (m, 1H), 4.20-4.40 (m, 1H), 4.07-4.18 (m, 1H), 4.00-4.07 (m, 1H), 3.80-3.95 (m, 1H), 3.40-3.70 (m, 3H), 1.90-2.30 (m, 14H). MS, m/z: 469.15 [M+H]+

### (3) Preparation of 1-O-(2,3,4,6-tetra-acetyl-D-glucoside)-butane-4,4-diethyl dicarboxylate (VI-2)

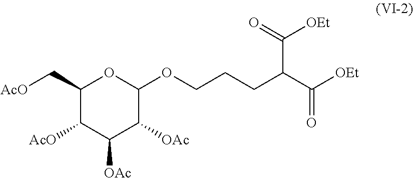 [SMILES: CCOC(=O)C(CCCOC1O[C@H](COC(C)=O)[C@@H](OC(C)=O)[C@H](OC(C)=O)[C@H]1OC(C)=O)C(=O)OCC]

2.1 g of 1-O-(2,3,4,6-tetra-acetyl-D-glucoside)-3-bromopropane (V-2) obtained in the previous step was dissolved in dry DMF (15 mL), to which was added potassium carbonate (2.5 g) followed by diethyl malonate (1.45 g), then the reaction mixture was stirred at room temperature overnight and the reaction was monitored by TLC. After completion of the reaction, ethyl acetate (100 mL) was added, washed with saturated NH4Cl (aq.) (1×50 mL), re-extracted with ethyl acetate (2×25 mL), then the organic phase was combined and washed with saturated NH4Cl (aq.) (1×100 mL), water (1×100 mL), brine (1×100 mL), dried over anhydrous sodium sulfate and concentrated under reduced pressure to give pale yellow crude product. The obtained crude product was purified by silica gel column chromatography (PE/EtOAc:3/1) to give the desired product as a colorless oil (VI-2). Yield: 2.2 g.

1H NMR (400 MHz, CDCl3): δ5.45 (t, J=9.6 Hz, 1H), 4.95-5.15 (m, 2H), 4.75-4.93 (m, 1H), 4.13-4.35 (m, 5H), 4.03-4.11 (m, 1H), 3.93-4.02 (m, 1H), 3.60-3.80 (m, 1H), 3.39-3.50 (m, 1H), 3.25-3.38 (t, J=8.0 Hz, 1H), 1.80-2.30 (m, 14H), 1.50-1.75 (m, 2H), 1.10-1.45 (m, 6H). MS, m/z: 549.50 [M+H]+

### (4) Preparation of 1-O-(2,3,4,6-tetra-acetyl-D-glucoside)-butane-4-chloro-4,4-diethyl dicarboxylate (VII-2)

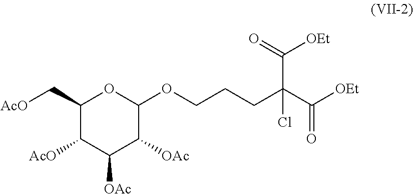 [SMILES: CCOC(=O)C(Cl)(CCCOC1O[C@H](COC(C)=O)[C@@H](OC(C)=O)[C@H](OC(C)=O)[C@H]1OC(C)=O)C(=O)OCC]

2.2 g of 1-O-(2,3,4,6-tetra-acetyl-D-glucoside)-butane-4,4-diethyl dicarboxylate was dissolved in dry THF (20 mL), and then cooled to 0° C. The air inside the flask was replaced with nitrogen, and to the above solution was slowly added solid sodium hydride (193 mg, 60% suspension in mineral oil) under a nitrogen atmosphere and stirred for 1 hour after warming to room temperature, then NCS (643 mg) was added and stirred for 2 hours at room temperature, the solvent was removed by rotary evaporation. Ethyl acetate (100 mL) was added, washed with saturated NH4Cl (aq.) (1×50 mL), re-extracted with ethyl acetate (2×25 mL), then the organic phase was combined and washed with saturated NH4Cl (aq.) (1×100 mL), water (1×100 mL), brine (1×100 mL), dried over anhydrous sodium sulfate and concentrated under reduced pressure to give the pale yellow crude product. The obtained crude product was purified by silica gel column chromatography (PE/EtOAc:3/1) to give the desired product as a colorless oil (VII-2). Yield: 2.2 g.

1H NMR (400 MHz, CDCl3): δ5.44 (t, J=6 Hz, 1H), 5.00-5.10 (m, 2H), 4.80-4.88 (m, 1H), 4.20-4.25 (m, 5H), 4.05-4.10 (m, 1H), 3.95-4.03 (m, 1H), 3.68-3.78 (m, 1H), 3.40-3.50 (m, 1H), 2.20-2.40 (m, 2H), 1.90-2.15 (m, 12H), 1.60-1.80 (m, 2H), 1.23-1.35 (m, 6H). MS, m/z: 583.19 [M+H]+

### (5) Preparation of 1-O-D-glucoside-butane-4-chloro-4,4-dicarboxylic acid (III-2)

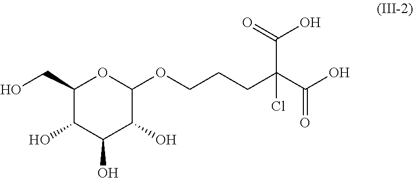 [SMILES: O=C(O)C(Cl)(CCCOC1O[C@H](CO)[C@@H](O)[C@H](O)[C@H]1O)C(=O)O]

1) 2.2 g of 1-O-(2,3,4,6-tetra-acetyl-D-glucoside)-butane-4-chloro-4,4-diethyl dicarboxylate was dissolved in methanol (5 mL), to which was then added a solution of sodium hydroxide (1.2 g) dissolved in water (10 mL) at room temperature and then heated at 60° C. for 24 hours. The reaction was monitored by TLC.

2) After completion of the reaction, the methanol was removed by rotary evaporation, then the residue was treated with strong acid cation exchange resin. The filtrate obtained from filtration of the resin was lyophilized to give a colorless viscous liquid (1.3 g). The crude product can be used directly in the next step. MS, m/z: 359.15 [M+H]+

### (6) Preparation of cis-[trans-(1R,2R)-diaminocyclohexane] Pt (II) (1-O-D-glycoside-butanee-4-chloro-4,4-dicarboxylate) (I-2)

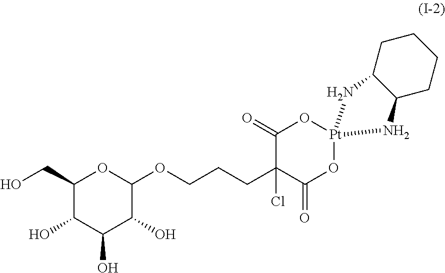

1) 1.3 g of crude 1-O-D-glucoside-butane-4-chloro-4,4-dicarboxylic acid was dissolved in water (15 mL), to which was added a freshly prepared aqueous solution of barium hydroxide to adjust the pH to 8, and then stirred at room temperature for 30 minutes.

2) Under the protection of nitrogen, to the reaction solution of 1) was added a solution of trans-(1R,2R)-diaminocyclohexane platinum sulfate (1.4 g) dissolved in water (2 mL) followed by a freshly prepared aqueous solution of barium hydroxide to re-adjust the pH to 8. The reaction mixture was stirred in the dark at a room temperature overnight.

3) After completion of the reaction, the precipitate was removed by centrifugation, the supernatant was collected and separated by semi-preparative HPLC, and the fraction of the collected product was collected and lyophilized by lyophilizer to obtain the final product (I-2) as a white solid. Yield: 1.5 g.

1H NMR (400 MHz, D2O): δ4.88 (d, J=3.6 Hz, 1H, α-isomer), 3.65-3.85 (m, 5H), 3.55-3.63 (m, 1H), 3.45-3.53 (m, 1H), 3.25-3.40 (m, 2H), 2.80-3.00 (m, 1H), 2.25-2.45 (m, 2H), 1.85-2.05 (m, 2H), 1.56-1.73 (m, 2H), 1.49 (d, J=8 Hz, 2H), 1.13-1.33 (m, 2H), 0.92-1.11 (m, 2H). MS, m/z: 666.65 [M+H]+

### Example 3

### Preparation of diamine Pt (II) (1-O-D-glucoside-propane-3-chloro-3,3-dicarboxylate) (I-3)

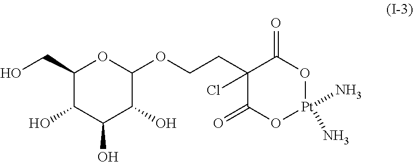

1) 100 mg of crude 1-O-D-glucoside-propane-3-chloro-3,3-dicarboxylic acid was dissolved in water (5 mL), to which was added a freshly prepared aqueous solution of barium hydroxide to adjust the pH to 9, and then stirred at room temperature for 30 minutes.

2) Under the protection of nitrogen, to the reaction solution of 1) was added a solution of diamine platinum sulfate (110 mg) dissolved in water (2 mL) followed by a freshly prepared aqueous solution of barium hydroxide to adjust the pH to 8. The reaction mixture was stirred in the dark at a room temperature overnight.

3) After completion of the reaction, the precipitate was removed by centrifugation, the supernatant was collected and separated by semi-preparative HPLC, and the fraction of the collected product was lyophilized by lyophilizer to obtain the final product (I-3) as a white solid. Yield: 110 mg.

1H NMR (400 MHz, D2O): δ4.88 (d, J=3.6 Hz, 0.8H, α-isomer), 4.42 (d, J=7.2 Hz, 0.2H, β-isomer), 3.15-3.95 (m, 10H). MS, m/z: 572.11 [M+H]+

### Example 4

### Preparation of diamine Pt (II) (1-O-D-glucoside-butane-4-chloro-4,4-dicarboxylate) (I-4)

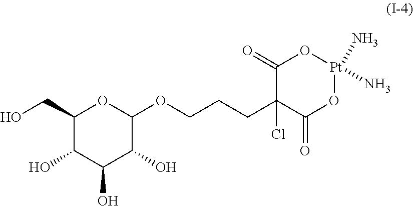

1) 100 mg of crude 1-O-D-glucoside-butane-4-chloro-4,4-dicarboxylic acid was dissolved in water (5 mL), to which was added a freshly prepared aqueous solution of barium hydroxide to adjust the pH to 9, and then stirred at room temperature for 30 minutes.

2) Under the protection of nitrogen, to the reaction solution of 1) was added a solution of diamine platinum sulfate (120 mg) dissolved in water (2 mL) followed by a freshly prepared aqueous solution of barium hydroxide to re-adjust the pH to 9. The reaction mixture was stirred in the dark at a room temperature overnight.

3) After completion of the reaction, the precipitate was removed by centrifugation, the supernatant was collected and separated by semi-preparative HPLC, and the fraction of the collected product was lyophilized by lyophilizer to obtain the final product (I-4) as a white solid. Yield: 110 mg.

1H NMR (400 MHz, D2O): δ4.87 (d, J=3.6 Hz, 1H, α-isomer), 3.64-3.83 (m, 5H), 3.55-3.63 (m, 1H), 3.43-3.53 (m, 1H), 3.26-3.40 (m, 2H), 2.80-2.98 (m, 1H), 1.60-1.75 (m, 2H). MS, m/z: 586.56 [M+H]+

### Example 5

### (1) Preparation of 1-O-D-galactoside-2-bromoethane (IV-5)

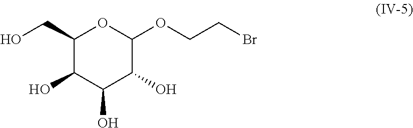 [SMILES: OC[C@H]1OC(OCCBr)[C@H](O)[C@@H](O)[C@H]1O]

1) To 2-bromoethanol (10 mL) was added galactose (2.7 g) at room temperature, and then cooled to 0° C., the air inside the flask was replaced with nitrogen, then 1 mL of BF3-Et2O complex was added dropwise under a nitrogen atmosphere;

2) The reaction solution was stirred at 0° C. for 15 minutes, then slowly warmed to room temperature and stirred for 30 minutes, then heated to 80° C. and stirred for 5 hours. After completion of the reaction, the solvent was evaporated in vacuum and the residue was simply subjected to purification on silica gel chromatography (CH2Cl2/CH3OH: 6/1) to give the crude product 2.4 g (IV-1). MS, m/z: 287.03 [M+H]+

### (2) Preparation of 1-O-(2,3,4,6-tetra-acetyl-D-galactoside)-2-bromoethane (V-5)

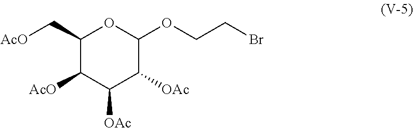 [SMILES: CC(=O)OC[C@H]1OC(OCCBr)[C@H](OC(C)=O)[C@@H](OC(C)=O)[C@H]1OC(C)=O]

2.4 g of 1-O-D-galactoside-2-bromoethane obtained in the previous step was dissolved in pyridine and acetic anhydride (7 mL:7 mL) at room temperature, then the reaction mixture was stirred overnight and the reaction was monitored by TLC. After completion of the reaction, ethyl acetate (100 mL) was added, washed with 5% (volume concentration) aqueous hydrochloric acid (2×25 mL), re-extracted with ethyl acetate (2×25 mL), then the organic phase was combined and washed with saturated NH4Cl (aq.) (1×100 mL), water (1×100 mL), saturated NaHCO3 (aq.) (1×100 mL), brine (1×100 mL), dried over anhydrous sodium sulfate and concentrated under reduced pressure to give the pale yellow crude product. The obtained crude product was purified by silica gel column chromatography (PE/EtOAc:3/1) to give the desired product as a white solid (V-5). Yield: 2.6 g.

1H NMR (400 MHz, CDCl3): δ5.46 (d, J=4 Hz, 1H), 5.33-5.45 (m, 1H), 5.19 (d, J=4 Hz, 1H), 5.07-5.15 (m, 1H), 4.33 (t, J=6 Hz, 1H), 4.06-4.13 (m, 2H), 3.95-4.05 (m, 1H), 3.77-3.88 (m, 1H), 3.51 (t, J=4 Hz, 2H), 1.95-2.20 (m, 12H). MS, m/z: 455.16 [M+H]+

### (3) Preparation of 1-O-(2,3,4,6-tetra-acetyl-D-galactoside) propane-3,3-diethyl dicarboxylate (VI-5)

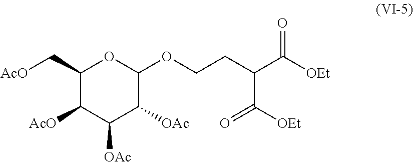 [SMILES: CCOC(=O)C(CCOC1O[C@H](COC(C)=O)[C@H](OC(C)=O)[C@H](OC(C)=O)[C@H]1OC(C)=O)C(=O)OCC]

2.6 g of 1-O-(2,3,4,6-tetra-acetyl-D-galactoside)-2-bromoethane (V-5) obtained in the previous step was dissolved in dry DMF (15 mL), to which was added potassium carbonate (3 g) followed by diethyl malonate (1.8 g), then the reaction mixture was stirred at room temperature overnight and the reaction was monitored by TLC. After completion of the reaction, ethyl acetate (100 mL) was added, washed with saturated NH4Cl (aq.) (1×50 mL), re-extracted with ethyl acetate (2×25 mL), then the organic phase was combined and washed with saturated NH4Cl (aq.) (1×100 mL), water (1×100 mL), brine (1×100 mL), dried over anhydrous sodium sulfate and concentrated under reduced pressure to give the pale yellow oil. The obtained crude product was purified by silica gel column chromatography (PE/EtOAc:3/1) to give the desired product as a white solid (VI-5). Yield: 2.8 g.

1H NMR (400 MHz, CDCl3): δ5.40 (d, J=4 Hz, 1H), 5.23-5.33 (m, 1H), 5.00-5.15 (m, 2H), 4.20-4.40 (m, 5H), 3.90-4.10 (m, 2H), 3.73-3.90 (m, 1H), 3.53-3.65 (m, 1H), 3.49 (t, J=4 Hz, 1H), 1.90-2.20 (m, 14H), 1.20-1.40 (m, 6H). MS, m/z: 535.26 [M+H]+

### (4) Preparation of 1-O-(2,3,4,6-tetra-acetyl-D-galactoside)-propane-3-chloro-3,3-dicarboxylate (VII-5)

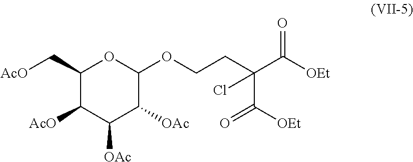 [SMILES: CCOC(=O)C(Cl)(CCOC1O[C@H](COC(C)=O)[C@H](OC(C)=O)[C@H](OC(C)=O)[C@H]1OC(C)=O)C(=O)OCC]

2.8 g of 1-O-(2,3,4,6-tetra-acetyl-D-galactoside)-propane-3,3-diethyl dicarboxylate was dissolved in dry THF (20 mL), and then cooled to 0° C. The air inside the flask was replaced with nitrogen, and to the above solution was slowly added solid sodium hydride (250 mg, 60% suspension in mineral oil) under a nitrogen atmosphere and stirred for 1 hour after warming to room temperature, then NCS (700 mg) was added and stirred for 2 hours at room temperature, the solvent was removed by rotary evaporation. Ethyl acetate (100 mL) was added, washed with saturated NH4Cl (aq.) (1×50 mL), re-extracted with ethyl acetate (2×25 mL), then the organic phase was combined and washed with saturated NH4Cl (aq.) (1×100 mL), water (1×100 mL), brine (1×100 mL), dried over anhydrous sodium sulfate and concentrated under reduced pressure to give the pale yellow oil. The obtained crude product was purified by silica gel column chromatography (PE/EtOAc:3/1) to give the desired product as a white solid (VII-5). Yield: 2.7 g.

1H NMR (400 MHz, CDCl3): δ5.42 (s, 1H), 5.25-5.33 (m, 1H), 5.03-5.10 (m, 2H), 4.18-4.40 (m, 5H), 4.00-4.15 (m, 2H), 3.83-3.95 (m, 1H), 3.45-3.58 (m, 1H), 2.52-2.60 (m, 1H), 2.45-2.50 (m, 1H), 1.90-2.20 (m, 12H), 1.25-1.35 (m, 6H). MS, m/z: 569.26 [M+H]+

### (5) Preparation of 1-O-D-galactoside-propane-3-chloro-3,3-dicarboxylic acid (III-5)

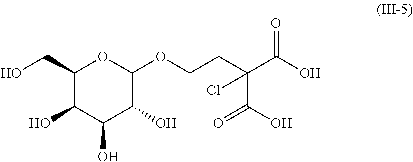 [SMILES: O=C(O)C(Cl)(CCOC1O[C@H](CO)[C@H](O)[C@H](O)[C@H]1O)C(=O)O]

1) 2.7 g of 1-O-(2,3,4,6-tetra-acetyl-D-galactoside)-propane-3-chloro-3,3-diethyl dicarboxylate was dissolved in methanol (5 mL), to which was then added a solution of sodium hydroxide (1.5 g) dissolved in water (10 mL) at room temperature and then heated at 60° C. for 24 hours. The reaction was monitored by TLC.

2) After completion of the reaction, the methanol was removed by rotary evaporation, then treated with strong acid cation exchange resin. The filtrate obtained by filtration of the resin was lyophilized to give a colorless viscous liquid (1.7 g) (III-5). The crude product was used directly in the next step. MS, m/z: 345.25 [M+H]+

### (6) Preparation of cis-[trans-(1R,2R)-diaminocyclohexane] Pt (II) (1-O-D-galactoside-propane-3-chloro-3,3-dicarboxylate) (I-5)

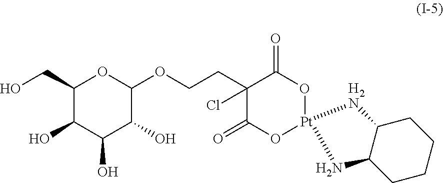

1) 1.6 g of crude 1-O-D-galactoside-propane-3-chloro-3,3-dicarboxylic acid was dissolved in water (15 mL), to which was added a freshly prepared aqueous solution of barium hydroxide to adjust the pH to 7, and then stirred at room temperature for 30 minutes.

2) Under the protection of nitrogen, to the reaction solution of 1) was added a solution of trans-(1R,2R)-diaminecyclohexane platinum sulfate (1.8 g) dissolved in water (5 mL) followed by a freshly prepared aqueous solution of barium hydroxide to re-adjust the pH to 7. The reaction mixture was stirred in the dark at a room temperature overnight.

3) After completion of the reaction, the precipitate was removed by centrifugation, the supernatant was collected and separated by semi-preparative HPLC, and the fraction of the collected product was lyophilized by a lyophilizer to obtain the final product (I-5) as a white solid. Yield: 1.7 g.

1H NMR (400 MHz, D2O): δ4.90 (d, J=3.6 Hz, 1H), 4.10-4.30 (m, 1H), 3.50-4.00 (m, 8H), 2.80-3.40 (m, 1H), 2.28-2.45 (m, 2H), 1.90-2.00 (m, 2H), 1.40-1.60 (m, 2H), 1.16-1.30 (br, 2H), 1.00-1.15 (m, 2H). MS, m/z: 652.33 [M+H]+

### Example 6

### (1) Preparation of 1-O-D-galactoside-3-bromopropane (IV-6)

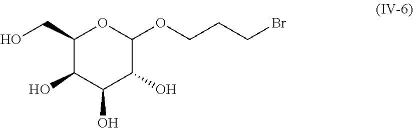 [SMILES: OC[C@H]1OC(OCCCBr)[C@H](O)[C@@H](O)[C@H]1O]

Following the method of Example 2 and replacing glucose with galactose produced the desired crude product (2.05 g) from 1.8 g of galactose. MS, m/z: 301.03 [M+H]+

### (2) Preparation of 1-O-(2,3,4,6-tetra-acetyl-D-galactoside)-3-bromo-propane (V-6)

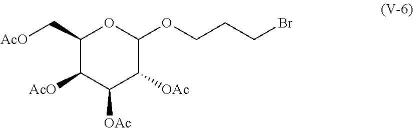 [SMILES: CC(=O)OC[C@H]1OC(OCCCBr)[C@H](OC(C)=O)[C@@H](OC(C)=O)[C@H]1OC(C)=O]

Following the method of Example 2 and replacing 1-O-D-glucoside-3-bromopropane with 1-O-D-galactoside-3-bromopropane produced the desired product (2.2 g) as a white solid d from 2.05 g of 1-O-D-galactoside-3-bromopropane. MS, m/z: 469.25 [M+H]+

(3) Preparation of 1-O-(2,3,4,6-tetra-acetyl-D-galactoside)-butane-4,4-diethyl dicarboxylate (VI-6)

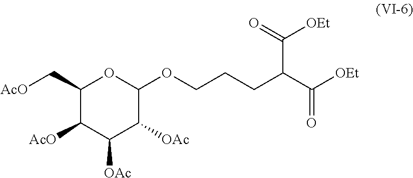 [SMILES: CCOC(=O)C(CCCOC1O[C@H](COC(C)=O)[C@H](OC(C)=O)[C@H](OC(C)=O)[C@H]1OC(C)=O)C(=O)OCC]

Following the method of Example 2 and replacing 1-O-(2,3,4,6-tetra-acetyl-D-glucoside)-3-bromopropane with 1-O-(2,3,4,6-tetra-acetyl-D-galactoside)-3-bromopropane provided the desired product (2.3 g) as a white solid from 2.2 g of 1-O-(2,3,4,6-tetra-acetyl-D-galactoside)-3-bromopropane. MS, m/z: 549.33 [M+H]+

### (4) Preparation of 1-O-(2,3,4,6-tetra-acetyl-D-galactoside)-butane-4-chloro-4,4-diethyl dicarboxylate (VII-6)

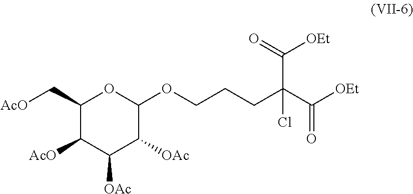 [SMILES: CCOC(=O)C(Cl)(CCCOC1O[C@H](COC(C)=O)[C@H](OC(C)=O)[C@H](OC(C)=O)[C@H]1OC(C)=O)C(=O)OCC]

Following the method of Example 2 and replacing 1-O-(2,3,4,6-tetra-acetyl-D-glucoside)-butane-4,4-diethyl dicarboxylate with 1-O-(2,3,4,6-tetra-acetyl-D-galactoside)-butane-4,4-diethyl dicarboxylate provided the desired product (2.3 g) as a white solid from 2.3 g of 1-O-(2,3,4,6-tetra-acetyl-D-galactoside)-butane-4,4-diethyl dicarboxylate. MS, m/z: 583.26 [M+H]+

### (5) Preparation of 1-O-D-galactoside-butane-4-chloro-4,4-dicarboxylic acid (III-6)

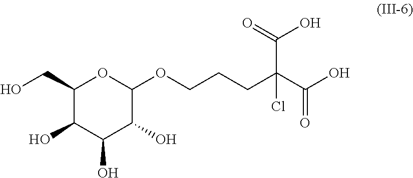 [SMILES: O=C(O)C(Cl)(CCCOC1O[C@H](CO)[C@H](O)[C@H](O)[C@H]1O)C(=O)O]

Following the method of Example 2 and replacing 1-O-(2,3,4,6-tetra-acetyl-D-glucoside)-butane-4-chloro-4,4-diethyl dicarboxylate with 1-O-(2,3,4,6-tetra-acetyl-D-galactoside)-butane-4-chloro-4,4-diethyl dicarboxylate provided the desired product (2.2 g) as a white solid from 2.3 g of 1-O-(2,3,4,6-tetra-acetyl-D-galactoside)-butane-4-chloro-4,4-diethyl dicarboxylate. MS, m/z: 359.26 [M+H]+

### (6) Preparation of cis-[trans-(1R,2R)-diaminocyclohexane] Pt (II) (1-O-D-galactoside-butane-4-chloro-4,4-dicarboxylate) (I-6)

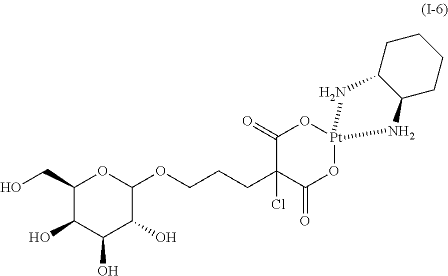

1) 1.3 g of crude 1-O-D-galactoside-butane-3-chloro-3,3-dicarboxylic acid was dissolved in water (15 mL), to which was added a freshly prepared aqueous solution of barium hydroxide to adjust the pH to 8, and then stirred at room temperature for 30 minutes.

2) Under the protection of nitrogen, to the reaction solution of 1) was added a solution of trans-(1R,2R)-diaminocyclohexane platinum sulfate (1.4 g) dissolved in water (2 mL) followed by a freshly prepared aqueous solution of barium hydroxide to re-adjust the pH to 8. The reaction mixture was stirred in the dark at a room temperature overnight.

3) After completion of the reaction, the precipitate was removed by centrifugation, the supernatant was collected and separated by semi-preparative HPLC, and the fraction of the collected product was lyophilized by a lyophilizer to obtain the final product (I-6) as a white solid. Yield; 1.4 g.

1H NMR (400 MHz, D2O): δ4.90 (d, J=4 Hz, 1H), 3.62-4.00 (m, 7H), 3.50-3.60 (m, 1H), 2.70-3.00 (m, 2H), 2.20-2.40 (m, 2H), 1.90-2.10 (m, 2H), 1.60-1.70 (m, 2H), 1.50 (d, J=6 Hz, 2H), 1.18-1.30 (m, 2H), 1.00-1.16 (m, 2H). MS, m/z: 666.20 [M+H]+

### Example 7

### Preparation of diamine Pt (II) (1-O-D-galactoside-propane-3-chloro-3,3-dicarboxylate) (I-7)

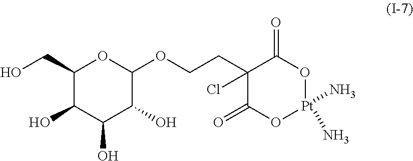

1) 100 mg of crude 1-O-D-galactoside-propane-3-chloro-3,3-dicarboxylic acid was dissolved in water (5 mL), to which was added a freshly prepared aqueous solution of barium hydroxide to adjust the pH to 9, and then stirred at room temperature for 30 minutes.

2) Under the protection of nitrogen, to the reaction solution of 1) was added a solution of diamine platinum sulfate (100 mg) dissolved in water (2 mL) followed by a freshly prepared aqueous solution of barium hydroxide to re-adjust the pH to 9. The reaction mixture was stirred in the dark at a room temperature overnight.

3) After completion of the reaction, the precipitate was removed by centrifugation, the supernatant was collected and separated by semi-preparative HPLC, and the fraction of the collected product was lyophilized by a lyophilizer to obtain the final product (I-7) as a white solid. Yield: 102 mg.

1H NMR (400 MHz, D2O): δ4.89 (d, J=3.6 Hz, 1H), 3.50-4.20 (m, 9H), 2.80-3.40 (m, 1H). MS, m/z: 572.21 [M+H]+

### Example 8

### Preparation of diamine Pt (II) (1-O-D-galactoside-butane-4-chloro-4,4-dicarboxylate) (I-8)

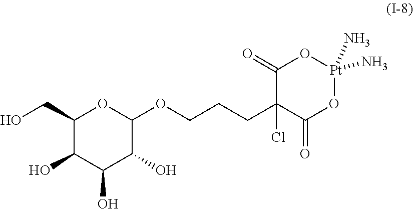

1) 100 mg of crude 1-O-D-galactoside-butane-4-chloro-4,4-dicarboxylic acid was dissolved in water (5 mL), to which was added a freshly prepared aqueous solution of barium hydroxide to adjust the pH to 9, and then stirred at room temperature for 30 minutes.

2) Under the protection of nitrogen, to the reaction solution of 1) was added a solution of diamine platinum sulfate (95 mg) dissolved in water (2 mL) followed by a freshly prepared aqueous solution of barium hydroxide to re-adjust the pH to 9. The reaction mixture was stirred in the dark at a room temperature overnight.

3) After completion of the reaction, the precipitate was removed by centrifuge, the supernatant was collected and separated by semi-preparative HPLC, and the fraction of the collected product was lyophilized by a lyophilizer to obtain the final product (I-8) as a white solid. Yield: 89 mg.

1H NMR (400 MHz, D2O): δ4.90 (d, J=4 Hz, 1H), 3.50-4.00 (m, 8H), 2.68-3.10 (m, 2H), 1.55-1.75 (m, 2H). MS, m/z: 586.19 [M+H]+

### Example 9

### (1) Preparation of 1-O-D-mannoside-2-bromoethane (IV-1)

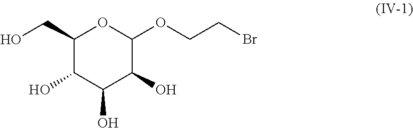 [SMILES: OC[C@H]1OC(OCCBr)[C@@H](O)[C@@H](O)[C@@H]1O]

1) To 2-bromoethanol (8 mL) was added mannose (1.8 g) at room temperature, and then cooled to 0° C., the air inside the flask was replaced with nitrogen, then 1 mL of BF3-Et2O complex was added dropwise under a nitrogen atmosphere;

2) The reaction solution was stirred at 0° C. for 15 minutes, then slowly warmed to room temperature and stirred for 30 minutes, then heated to 80° C. and stirred for 5 hours; After completion of the reaction, the solvent was evaporated in vacuum and the residue was simply subjected to purification on silica gel chromatography (CH2Cl2/CH3OH: 6/1) to give the crude product (IV-1). Yield: 2.1 g. MS, m/z: 287. 05 [M+H]+

### (2) Preparation of 1-O-(2,3,4,6-tetra-acetyl-D-mannoside)-2-bromoethane (V-9)

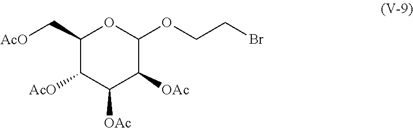 [SMILES: CC(=O)OC[C@H]1OC(OCCBr)[C@@H](OC(C)=O)[C@@H](OC(C)=O)[C@@H]1OC(C)=O]

2.1 g of 1-O-D-mannoside-2-bromoethane (IV-9) obtained in the previous step was dissolved in pyridine and acetic anhydride (7 mL:7 mL) at room temperature, then the reaction mixture was stirred overnight and the reaction was monitored by TLC. After completion of the reaction, ethyl acetate (100 mL) was added, washed with 5% (volume concentration) aqueous hydrochloric acid (2×25 mL), re-extracted with ethyl acetate (2×25 mL), then the organic phase was combined and washed with saturated NH4Cl (aq.) (1×100 mL), water (1×100 mL), saturated NaHCO3 (aq.) (1×100 mL), brine (1×100 mL), dried over anhydrous sodium sulfate and concentrated under reduced pressure to give the pale yellow crude product. The obtained crude product was purified by silica gel column chromatography (PE/EtOAc:3/1) to give the desired product as a white solid (V-9). Yield: 2 g.

1H NMR (400 MHz, CDCl3): δ5.18-5.40 (m, 3H), 4.90 (s, 1H), 4.20-4.40 (m, 1H), 4.08-4.18 (m, 2H), 3.95-4.05 (m, 1H), 3.80-3.94 (m, 1H), 3.53 (t, J=6 Hz, 2H), 1.95-2.20 (m, 12H) MS, m/z: 455.09 [M+H]+

### (3) Preparation of 1-O-(2,3,4,6-tetra-acetyl-D-mannoside)-propane-3,3-diethyl dicarboxylate (VI-9)

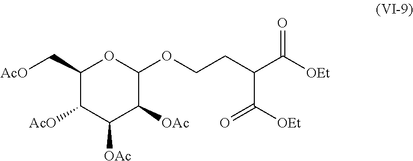 [SMILES: CCOC(=O)C(CCOC1O[C@H](COC(C)=O)[C@@H](OC(C)=O)[C@H](OC(C)=O)[C@@H]1OC(C)=O)C(=O)OCC]

2 g of 1-O-(2,3,4,6-tetra-acetyl-D-mannoside)-2-bromoethane (VI-9) obtained in the previous step was dissolved in dry DMF (5 mL), to which was added potassium carbonate (2.4 g) followed by diethyl malonate (1.4 g), then the reaction mixture was stirred at room temperature overnight and the reaction was monitored by TLC. After completion of the reaction, ethyl acetate (100 mL) was added, washed with saturated NH4Cl (aq.) (1×50 mL), re-extracted with ethyl acetate (2×25 mL), then the organic phase was combined and washed with saturated NH4Cl (aq.) (1×100 mL), water (1×100 mL), brine (1×100 mL), dried over anhydrous sodium sulfate and concentrated under reduced pressure to give the pale yellow crude product. The obtained crude product was purified by silica gel column chromatography (PE/EtOAc:3/1) to give the desired product as a white solid (VI-9). Yield: 2.1 g.

1H NMR (400 MHz, CDCl3): δ5.28 (d, J=4 Hz, 2H), 5.20-5.25 (m, 1H), 4.79 (s, 1H), 4.15-4.35 (m, 5H), 4.05-4.13 (m, 1H), 3.90-4.03 (m, 1H), 3.70-3.85 (m, 1H), 3.40-3.58 (m, 2H), 2.17-2.35 (m, 2H), 1.95-2.15 (m, 12H), 1.20-1.35 (m, 6H). MS, m/z: 535.18[M+H]+

### (4) Preparation of 1-O-(2,3,4,6-tetra-acetyl-D-mannoside)-propane-3-chloro-3,3-diethyl dicarboxylate (VII-9)

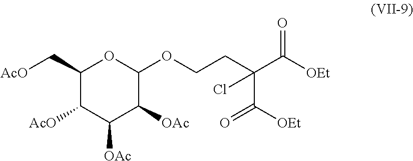 [SMILES: CCOC(=O)C(Cl)(CCOC1O[C@H](COC(C)=O)[C@@H](OC(C)=O)[C@H](OC(C)=O)[C@@H]1OC(C)=O)C(=O)OCC]

2.1 g of 1-O-(2,3,4,6-tetra-acetyl-D-mannoside)-propane-3,3-diethyl dicarboxylate was dissolved in dry THF (20 mL), and then cooled to 0° C. The air inside the flask was replaced with nitrogen, and to the above solution was slowly added solid sodium hydride (188 mg, 60% suspension in mineral oil) under a nitrogen atmosphere and stirred for 1 hour after warming to room temperature, then NCS (630 mg) was added and stirred for 2 hours at room temperature, the solvent was removed by rotary evaporation. Ethyl acetate (100 mL) was added, washed with saturated NH4Cl (aq.) (1×50 mL), re-extracted with ethyl acetate (2×25 mL), then the organic phase was combined and washed with saturated NH4Cl (aq.) (1×100 mL), water (1×100 mL), brine (1×100 mL), dried over anhydrous sodium sulfate and concentrated under reduced pressure to give the pale yellow crude product. The obtained crude product was purified by silica gel column chromatography (PE/EtOAc:3/1) to give the desired product as a white solid (VII-9). Yield: 2.1 g.

1H NMR (400 MHz, CDCl3): δ5.26 (t, J=8 Hz, 1H), 5.10-5.23 (m, 2H), 4.73 (s 1H), 4.25-4.40 (m, 5H), 4.00-4.15 (m, 3H), 3.55-3.65 (m, 1H), 2.63-2.75 (m, 1H), 2.50-2.60 (m, 1H), 1.90-2.20 (m, 12H), 1.25-1.35 (m, 6H). MS, m/z: 569.20 [M+H]+

### (5) Preparation of 1-O-D-mannoside-propane-3-chloro-3,3-dicarboxylic acid (III-9)

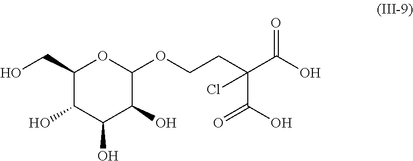 [SMILES: O=C(O)C(Cl)(CCOC1O[C@H](CO)[C@@H](O)[C@H](O)[C@@H]1O)C(=O)O]

1) 2.1 g of 1-O-(2,3,4,6-tetra-acetyl-D-mannoside)-propane-3-chloro-3,3-diethyl dicarboxylate (VII-9) was dissolved in methanol (5 mL), to which was then added a solution of sodium hydroxide (1.2 g) dissolved in water (10 mL) at room temperature and then heated at 60° C. for 24 hours. The reaction was monitored by TLC.

2) After completion of the reaction, the methanol was removed by rotary evaporation, then treated with strong acid cation exchange resin. The filtrate obtained from the filtration of the resin was lyophilized to give a colorless viscous liquid (1.1 g). The crude product was used directly in the next step. MS, m/z: 345.19 [M+H]+

### (6) Preparation of cis-[trans-(1R,2R)-diaminocyclohexane] Pt (II) (1-O-D-mannoside-propane-3-chloro-3,3-dicarboxylate) (I-9)

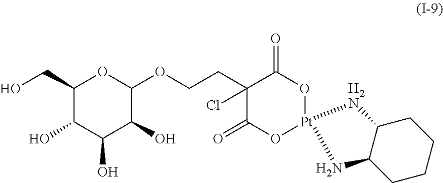

1) 1.1 g of crude 1-O-D-mannoside-propane-3-chloro-3,3-dicarboxylic acid was dissolved in water (15 mL), to which was added a freshly prepared aqueous solution of barium hydroxide to adjust the pH to 7, and then stirred at room temperature for 30 minutes.

2) Under the protection of nitrogen, to the reaction solution of 1) was added a solution of trans-(1R,2R)-diaminocyclohexane platinum sulfate (1.2 g) dissolved in water (5 mL) followed by a freshly prepared aqueous solution of barium hydroxide to re-adjust the pH to 7. The reaction mixture was stirred in the dark at a room temperature overnight.

3) After completion of the reaction, the precipitate was removed by centrifugation, the supernatant was collected and separated by semi-preparative HPLC, and the fraction of the collected product was lyophilized by a lyophilizer to obtain the final product (I-9) as a white solid. Yield: 1.5 g.

1H NMR (400 MHz, D2O): δ4.89 (s, 1H), 3.30-4.00 (m, 9H), 2.90-3.20 (m, 1H), 2.20-2.45 (m, 2H) 1.90-2.05 (m, 2H), 1.50 (d, J=8 Hz, 2H), 1.16-1.30 (m, 2H), 1.00-1.15 (m, 2H). MS, m/z: 652.16 [M+H]+

### Example 10

### (1) Preparation of 1-O-D-mannoside-3-bromopropane (IV-10)

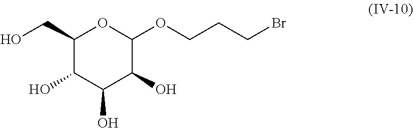 [SMILES: OC[C@H]1OC(OCCCBr)[C@@H](O)[C@@H](O)[C@@H]1O]

1) To 3-bromoethanol (10 mL) was added mannose (1.8 g) at room temperature, and then cooled to 0° C., the air inside the flask was replaced with nitrogen, then 1 mL of BF3-Et2O complex was added dropwise under a nitrogen atmosphere;

2) The reaction solution was stirred at 0° C. for 15 minutes, then slowly warmed to room temperature and stirred for 30 minutes, then heated to 80° C. and stirred for 5 hours. After completion of the reaction, the solvent was evaporated in vacuum and the residue was simply subjected to purification on silica gel chromatography (CH2Cl2/CH3OH: 6/1) to give the crude product (IV-2). Yield: 2 g. MS, m/z: 301.11[M+H]+

### (2) Preparation of 1-O-(2,3,4,6-tetra-acetyl-D-mannoside)-3-bromopropane (V-10)

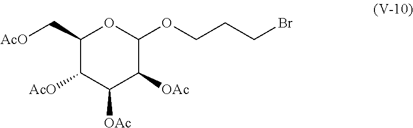 [SMILES: CC(=O)OC[C@H]1OC(OCCCBr)[C@@H](OC(C)=O)[C@@H](OC(C)=O)[C@@H]1OC(C)=O]

2 g of 1-O-D-mannoside-3-bromoethane (V-2) obtained in the previous step was dissolved in pyridine and acetic anhydride (6 mL:6 mL) at room temperature, then the reaction mixture was stirred overnight and the reaction was monitored by TLC. After completion of the reaction, ethyl acetate (100 mL) was added, washed with 5% (volume concentration) aqueous hydrochloric acid (2×25 mL), re-extracted with ethyl acetate (2×25 mL), then the organic phase was combined and washed with saturated NH4Cl (aq.) (1×100 mL), water (1×100 mL), saturated NaHCO3 (aq.) (1×100 mL), brine (1×100 mL), dried over anhydrous sodium sulfate and concentrated under reduced pressure to give pale yellow crude product. The obtained crude product was purified by silica gel column chromatography (PE/EtOAc:3/1) to give the desired product as a white solid (V-10). Yield: 2.3 g.

1H NMR (400 MHz, CDCl3): δ5.30-5.55 (m, 3H), 4.93 (s, 1H), 4.20-4.38 (m, 1H), 4.05-4.18 (m, 1H), 4.00-4.04 (m, 1H), 3.81-3.95 (m, 1H), 345-3.70 (m, 3H), 1.90-2.30 (m, 14H). MS, m/z: 469.16 [M+H]+

### (3) Preparation of 1-O-(2,3,4,6-tetra-acetyl-D-mannoside)-butane-3,3-diethyl dicarboxylate (VI-10)

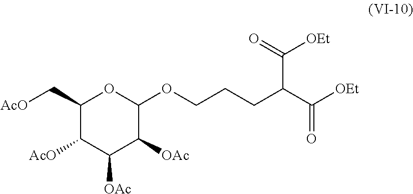 [SMILES: CCOC(=O)C(CCCOC1O[C@H](COC(C)=O)[C@@H](OC(C)=O)[C@H](OC(C)=O)[C@@H]1OC(C)=O)C(=O)OCC]

2.3 g of 1-O-(2,3,4,6-tetra-acetyl-D-mannoside)-3-bromoethane (V-10) obtained in the previous step was dissolved in dry N,N-dimethyl formamide (15 mL), to which was added potassium carbonate (2.7 g) followed by diethyl malonate (1.5 g), then the reaction mixture was stirred at room temperature overnight and the reaction was monitored by TLC. After completion of the reaction, ethyl acetate (100 mL) was added, washed with saturated NH4Cl (aq.) (1×50 mL), re-extracted with ethyl acetate (2×25 mL), then the organic phase was combined and washed with saturated NH4Cl (aq.) (1×100 mL), water (1×100 mL), brine (1×100 mL), dried over anhydrous sodium sulfate and concentrated under reduced pressure to give the pale yellow crude product. The obtained crude product was purified by silica gel column chromatography (PE/EtOAc:3/1) to give the desired product as a white solid (VI-10). Yield: 2.4 g.

1H NMR (400 MHz, CDCl3): δ5.15-5.40 (m, 3H), 4.78 (s, 1H), 4.25-4.36 (m, 1H), 4.15-4.19 (m, 4H), 4.05-4.13 (m, 1H), 3.90-4.03 (m, 1H), 3.65-3.80 (m, 1H), 3.43-3.58 (m, 1H), 3.36 (t, J=6 Hz, 1H), 1.88-2.20 (m, 14H), 1.60-1.75 (m, 2H), 1.10-1.35 (m, 6H). MS, m/z: 549.23 [M+H]+

### (4) Preparation of 1-O-(2,3,4,6-tetra-acetyl-D-mannoside)-butane-4-chloro-4,4-diethyl dicarboxylate (VII-10)

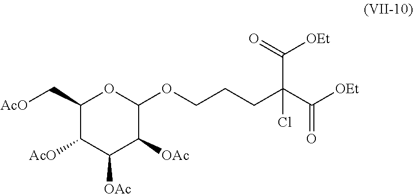 [SMILES: CCOC(=O)C(Cl)(CCCOC1O[C@H](COC(C)=O)[C@@H](OC(C)=O)[C@H](OC(C)=O)[C@@H]1OC(C)=O)C(=O)OCC]

2.4 g of 1-O-(2,3,4,6-tetra-acetyl-D-mannoside)-butane-4,4-diethyl dicarboxylate was dissolved in dry tetrahydrofuran (20 mL), and then cooled to 0° C. The air inside the flask was replaced with nitrogen, and to the above solution was slowly added solid sodium hydride (210 mg, 60% suspension in mineral oil) under a nitrogen atmosphere and stirred for 1 hour after warming to room temperature, then NCS (700 mg) was added and stirred for 2 hours at room temperature, the solvent was removed by rotary evaporation. Ethyl acetate (100 mL) was added, washed with saturated NH4Cl (aq.) (1×50 mL), re-extracted with ethyl acetate (2×25 mL), then the organic phase was combined and washed with saturated NH4Cl (aq.) (1×100 mL), water (1×100 mL), brine (1×100 mL), dried over anhydrous sodium sulfate and concentrated under reduced pressure to give the pale yellow crude product. The obtained crude product was purified by silica gel column chromatography (PE/EtOAc: 3/1) to give the desired product as white solid (VI-10). Yield: 2.3 g.

1H NMR (400 MHz, CDCl3): δ5.20-5.40 (m, 3H), 4.83 (s, 1H), 4.20-4.40 (m, 5H), 4.05-4.15 (m, 1H), 3.93-4.03 (m, 1H), 3.65-3.80 (m, 1H), 3.45-3.58 (m, 1H), 2.27-2.38 (m, 2H), 1.95-2.20 (m, 12H), 1.65-1.85 (m, 2H), 1.25-1.40 (m, 6H). MS, m/z: 583.23 [M+H]+

### (5) Preparation of 1-O-D-mannoside-butane-4-chloro-4,4-dicarboxylic acid (III-10)

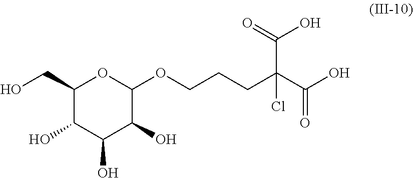 [SMILES: O=C(O)C(Cl)(CCCOC1O[C@H](CO)[C@@H](O)[C@H](O)[C@@H]1O)C(=O)O]

1) 2.3 g of 1-O-(2,3,4,6-tetra-acetyl-D-glucoside)-butane-4-chloro-4,4-diethyl dicarboxylate was dissolved in methanol (5 mL), to which was added a solution of sodium hydroxide (1.5 g) dissolved in 10 mL water at room temperature and then heated at 60° C. for 24 hours. The reaction was monitored by TLC.

2) After completion of the reaction, the methanol was removed rotary evaporation, then treated with strong acid cation exchange resin. The filtrate obtained from the filtration of the resin was lyophilized to give a colorless viscous liquid (1.3 g), the crude product was used directly in the next step. MS, m/z: 359.13 [M+H]+

### (6) Preparation of cis-[trans-(1R,2R)-diaminocyclohexane] Pt (II) (1-O-D-mannoside-butane-4-chloro-4,4-dicarboxylate) (I-10)

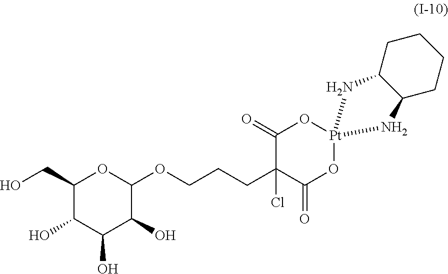

1) 1.3 g of crude 1-O-D-mannoside-butane-3-chloro-3,3-dicarboxylic acid was dissolved in water (15 mL), to which was added a freshly prepared aqueous solution of barium hydroxide to adjust the pH to 8, and then stirred at room temperature for 30 minutes.

2) Under the protection of nitrogen, to the reaction solution of 1) was added a solution of trans-(1R,2R)-diaminocyclohexane platinum sulfate (1.5 g) dissolved in water (2 mL) followed by a freshly prepared aqueous solution of barium hydroxide to re-adjust the pH to 8. The reaction mixture was stirred in the dark at a room temperature overnight.

3) After completion of the reaction, the precipitate was removed by centrifugation, the supernatant was collected and separated by semi-preparative HPLC, and the fraction of the collected product was lyophilized by a lyophilizer to obtain the final product (I-10) as a white solid. Yield: 1.4 g.

1H NMR (400 MHz, D20): δ4.86 (s, 1H), 3.50-3.96 (m, 8H), 2.80-3.20 (m, 2H), 2.20-2.45 (m, 2H), 1.96 (d, J=12 Hz, 2H), 1.61-1.75 (m, 2H), 1.51 (d, J=6 Hz, 2H), 1.13-1.30 (m, 2H), 0.95-1.12 (m, 2H). MS, m/z: 666.18 [M+H]+

### Example 11

### Preparation of diamine Pt (II) (1-O-D-mannoside-propane-3-chloro-3,3-dicarboxylate) (I-11)

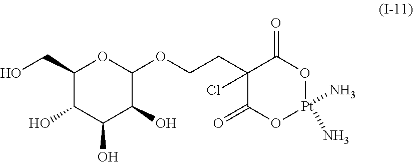

1) 100 mg of crude 1-O-D-mannoside-propane-3-chloro-3,3-dicarboxylic acid was dissolved in water (5 mL), to which was added a freshly prepared aqueous solution of barium hydroxide to adjust the pH to 9, and then stirred at room temperature for 30 minutes.

2) Under the protection of nitrogen, to the reaction solution of 1) was added a solution of diamine platinum sulfate (100 mg) dissolved in water (2 mL) followed by a freshly prepared aqueous solution of barium hydroxide to re-adjust the pH to 9. The reaction mixture was stirred in the dark at a room temperature overnight.

3) After completion of the reaction, the precipitate was removed by centrifugation, the supernatant was collected and separated by semi-preparative HPLC, and the fraction of the collected product was lyophilized by a lyophilizer to obtain the final product (I-11) as a white solid. Yield: 90 mg.

1H NMR (400 MHz, D20): δ4.85 (s, 1H), 3.50-3.95 (m, 9H), 2.80-3.20 (m, 1H). MS, m/z: 572.21 [M+H]+

### Example 12

### Preparation of diamine Pt (II) (1-O-D-mannoside-butane-4-chloro-4,4-dicarboxylate) (I-12)

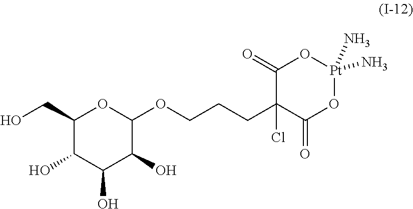

1) 100 mg of crude 1-O-D-mannoside-butane-4-chloro-4,4-dicarboxylic acid was dissolved in water (5 mL), to which was added a freshly prepared aqueous solution of barium hydroxide to adjust the pH to 9, and then stirred at room temperature for 30 minutes.

2) Under the protection of nitrogen, to the reaction solution of 1) was added a solution diamine platinum sulfate (90 mg) dissolved in water (2 mL) followed by a freshly prepared aqueous solution of barium hydroxide to re-adjust the pH to 9. The reaction mixture was stirred in the dark at a room temperature overnight.

3) After completion of the reaction, the precipitate was removed by centrifugation, the supernatant was collected and separated by semi-preparative HPLC, and the fraction of the collected product was lyophilized by a lyophilizer to obtain the final product (I-12) as a white solid. Yield: 80 mg.

1H NMR (400 MHz, D20): δ4.90 (s, 1H), 3.50-4.00 (m, 8H), 2.80-3.20 (m, 2H), 1.60-1.73 (m, 2H). MS, m/z: 586.17 [M+H]+

### Example 13

### Preparation of bisdiisopropylamine platinum Pt (II) (1-O-D-glucoside-propane-3-chloro-3,3-dicarboxylate) (I-13)

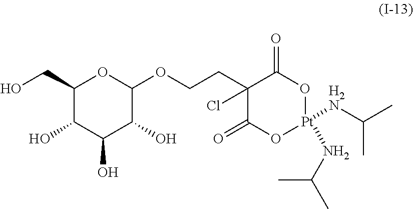

1) 100 mg of crude 1-O-D-glucoside-propane-3-chloro-3,3-dicarboxylic acid was dissolved in water (5 mL), to which was added a freshly prepared aqueous solution of barium hydroxide in water to adjust the pH to 9, and then stirred at room temperature for 30 minutes.

2) Under the protection of nitrogen, to the reaction solution of 1) was added a solution of bisdiisopropylamine platinum sulfate (140 mg) dissolved in water (2 mL) followed by a freshly prepared aqueous solution of barium hydroxide to re-adjust the pH to 9. The reaction mixture was stirred in the dark at a room temperature overnight.

3) After completion of the reaction, the precipitate was removed by centrifugation, the supernatant was collected and separated by semi-preparative HPLC, and the fraction of the collected product was lyophilized by a lyophilizer to obtain the final product (I-13) as a white solid. Yield: 90 mg.

1H NMR (400 MHz, D20): δ4.85 (s, 1H), 3.40-4.10 (m, 9H), 2.95-3.20 (m, 1H), 1.60-1.73 (m, 2H). MS, m/z: 656.21 [M+H]+

### Example 14

### Preparation of bisdiisopropylamine platinum Pt (II) (1-O-D-glucoside-butane-4-chloro-4,4-dicarboxylate) (I-14)

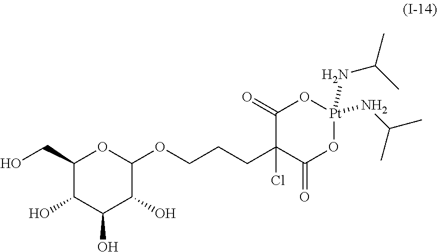

1) 100 mg of crude 1-O-D-glucoside-butane-4-chloro-4,4-dicarboxylic acid was dissolved in water (5 mL), to which was added a freshly prepared aqueous solution of barium hydroxide to adjust the pH to 9, and then stirred at room temperature for 30 minutes.

2) Under the protection of nitrogen, to the reaction solution of 1) was added a solution of bisdiisopropylamine platinum sulfate (150 mg) dissolved in water (2 mL) followed by a freshly prepared aqueous solution of barium hydroxide to re-adjust the pH to 9. The reaction mixture was stirred in the dark at a room temperature overnight.

3) After completion of the reaction, the precipitate was removed by centrifugation, the supernatant was collected and separated by semi-preparative HPLC, and the fraction of the collected product was lyophilized by a lyophilizer to obtain the final product (I-13) as a white solid. Yield: 90 mg.

1H NMR (400 MHz, D20): δ4.85 (s, 1H), 3.40-4.10 (m, 9H), 2.95-3.20 (m, 1H). MS, m/z: 670.28 [M+H]+

### Example 15

### Preparation of bisdiisopropylamine platinum Pt (II) (1-O-D-galactoside-propane-3-chloro-3,3-dicarboxylate) (I-15)

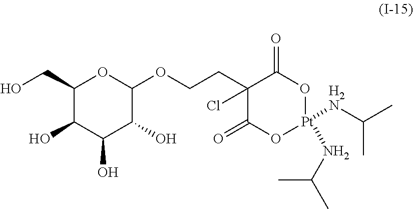

1) 100 mg of crude 1-O-D-galactoside-propane-3-chloro-3,3-dicarboxylic acid was dissolved in water (5 mL), to which was added a freshly prepared aqueous solution of barium hydroxide to adjust the pH to 9, and then stirred at room temperature for 30 minutes.

2) Under the protection of nitrogen, to the reaction solution of 1) was added a solution of bisdiisopropylamine platinum sulfate (140 mg) dissolved in water (2 mL) followed by a freshly prepared aqueous solution of barium hydroxide in water to re-adjust the pH to 9. The reaction mixture was stirred in the dark at a room temperature overnight.

3) After completion of the reaction, the precipitate was removed by centrifugation, the supernatant was collected and separated by semi-preparative HPLC, and the fraction of the collected product was lyophilized by a lyophilizer to obtain the final product (I-15) as a white solid. Yield: 91 mg.

1H NMR (400 MHz, D20): δ4.85 (s, 1H), 3.40-4.10 (m, 9H), 2.95-3.20 (m, 1H). MS, m/z: 656.23 [M+H]+

### Example 16

### Preparation of bisdiisopropylamine platinum Pt (II) (1-O-D-galactoside-butane-4-chloro-4,4-dicarboxylate) (I-16)

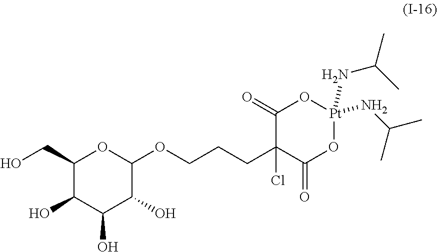

1) 100 mg of crude 1-O-D-galactoside-butane-4-chloro-4,4-dicarboxylic acid was dissolved in water (5 mL), to which was added a freshly prepared aqueous solution of barium hydroxide to adjust the pH to 9, and then stirred at room temperature for 30 minutes.

2) Under the protection of nitrogen, to the reaction solution of 1) was added a solution of bisdiisopropylamine platinum sulfate (150 mg) dissolved in water (2 mL) followed by a freshly prepared aqueous solution of barium hydroxide to adjust the pH to 9. The reaction mixture was stirred in the dark at a room temperature overnight.

3) After completion of the reaction, the precipitate was removed by centrifugation, the supernatant was collected and separated by semi-preparative HPLC, and the fraction of the collected product was lyophilized by a lyophilizer to obtain the final product (I-16) as a white solid. Yield: 113 mg.

1H NMR (400 MHz, D20): δ4.85 (s, 1H), 3.40-4.10 (m, 9H), 2.95-3.20 (m, 1H). MS, m/z: 670.21 [M+H]+

### Example 17

### Preparation of bisdiisopropylamine platinum Pt (II) (1-O-D-mannoside-propane-3-chloro-3,3-dicarboxylate) (I-17)

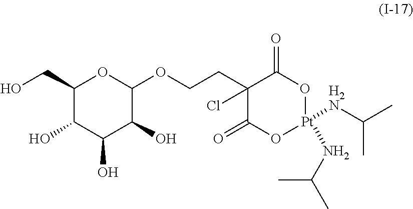

1) 100 mg of crude 1-O-D-mannoside-propane-3-chloro-3,3-dicarboxylic acid was dissolved in water (5 mL), to which was added a freshly prepared aqueous solution of barium hydroxide to adjust the pH to 9, and then stirred at room temperature for 30 minutes.

2) Under the protection of nitrogen, to the reaction solution of 1) was added a solution of bisdiisopropylamine platinum sulfate (140 mg) dissolved in water (2 mL) followed by a freshly prepared aqueous solution of barium hydroxide to re-adjust the pH to 9. The reaction mixture was stirred in the dark at a room temperature overnight.

3) After completion of the reaction, the precipitate was removed by centrifugation, the supernatant was collected and separated by semi-preparative HPLC, and the fraction of the collected product was lyophilized by a lyophilizer to obtain the final product (I-17) as a white solid. Yield: 97 mg.

1H NMR (400 MHz, D20): δ4.85 (s, 1H), 3.40-4.10 (m, 9H), 2.95-3.20 (m, 1H). MS, m/z: 656.21 [M+H]+

### Example 18

### Preparation of bisdiisopropylamine platinum Pt (II) (1-O-D-mannoside-butane-4-chloro-4,4-dicarboxylate) (I-18)

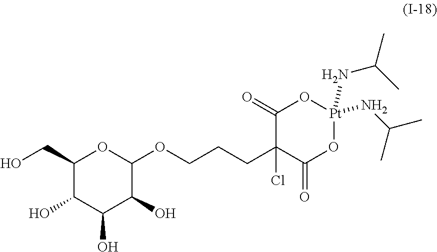

1) 100 mg of crude 1-O-D-mannoside-butane-4-chloro-4,4-dicarboxylic acid was dissolved in water (5 mL), to which was added a freshly prepared aqueous solution of barium hydroxide to adjust the pH to 9, and then stirred at room temperature for 30 minutes.

2) Under the protection of nitrogen, to the reaction solution of 1) was added a solution of bisdiisopropylamine platinum sulfate (150 mg) dissolved in water (2 mL) followed by a freshly prepared aqueous solution of barium hydroxide to re-adjust the pH to 9. The reaction mixture was stirred in the dark at a room temperature overnight.

3) After completion of the reaction, the precipitate was removed by centrifugation, the supernatant was collected and separated by semi-preparative HPLC, and the fraction of the collected product was lyophilized by a lyophilizer to obtain the final product (I-18) as a white solid. Yield: 116 mg.

1H NMR (400 MHz, D20): δ4.85 (s, 1H), 3.40-4.10 (m, 9H), 2.95-3.20 (m, 1H). MS, m/z: 670.28 [M+H]+

### Example 19

### (1) Preparation of 1-O-(2,3,4,6-tetra-acetyl-D-glucoside)-ethane-2,2-diethyl dicarboxylate (VI-19)

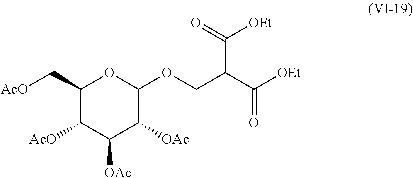 [SMILES: CCOC(=O)C(COC1O[C@H](COC(C)=O)[C@@H](OC(C)=O)[C@H](OC(C)=O)[C@H]1OC(C)=O)C(=O)OCC]

1) To the 1-hydroxy-ethane-2,2-diethyl dicarboxylate (5 mL) (prepared according to the literature Kogyo Kagaku Zasshi, 1954, vol. 57, p. 140) was added acetylated glucose (2.7 g) at room temperature and then cooled to 0° C., the air inside the flask was replaced with nitrogen, then 1 mL of BF3-Et2O complex was added dropwise under a nitrogen atmosphere.

2) The reaction solution was stirred at 0° C. for 15 minutes, then slowly warmed to room temperature and stirred for 30 minutes, then heated to 60° C. and stirred for 5 hours; After completion of the reaction, the solvent was evaporated in vacuum and the residue was simply subjected to purification on silica gel chromatography to give the crude product (VI-19). Yield: 3.3 g. MS, m/z: 521.25 [M+H]+

### (2) Preparation of 1-O-(2,3,4,6-tetra-acetyl-D-glucoside)-ethane-2-chloro-2,2-diethyl dicarboxylate (VII-19)

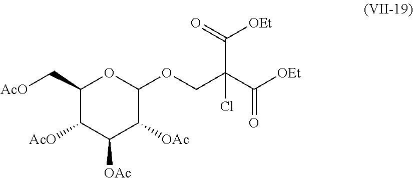 [SMILES: CCOC(=O)C(Cl)(COC1O[C@H](COC(C)=O)[C@@H](OC(C)=O)[C@H](OC(C)=O)[C@H]1OC(C)=O)C(=O)OCC]

3.0 g of 1-O-(2,3,4,6-tetra-acetyl-D-glucoside)-ethane-2,2-diethyl dicarboxylate was dissolved in dry THF (20 mL), and then cooled to 0° C. The air inside the flask was replaced with nitrogen, and to the above solution was slowly added solid sodium hydride (235 mg, 60% suspension in mineral oil) under a nitrogen atmosphere and stirred for 12 hours after warming to room temperature, then 1.5 equivalents of NCS was added and stirred for 4 hours at room temperature, the solvent was removed by rotary evaporation. Ethyl acetate (100 mL) was added, washed with saturated NH4Cl (aq.) (1×50 mL), re-extracted with ethyl acetate (2×25 mL), then the organic phase was combined and washed with saturated NH4Cl (aq.) (1×100 mL), water (1×100 mL), brine (1×100 mL), dried over anhydrous sodium sulfate and concentrated under reduced pressure to give the pale yellow crude product. The obtained crude product was purified by silica gel column chromatography (PE/EtOAc: 3/1) to give the desired product as a colorless oil (VII-19). Yield: 2.6 g. MS, m/z: 555.203 [M+H]+

### (3) Preparation of 1-O-D-glucoside-ethane-2-chloro-2,2-dicarboxylic acid (III-19)

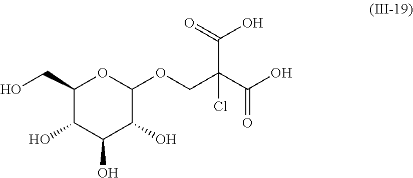 [SMILES: O=C(O)C(Cl)(COC1O[C@H](CO)[C@@H](O)[C@H](O)[C@H]1O)C(=O)O]

1) 1.3 g of 1-O-(2,3,4,6-tetra-acetyl-D-glucoside)-ethane-2-chloro-2,2-diethyl dicarboxylate was dissolved in methanol (5 mL), to which was added a solution of sodium hydroxide (1 g) dissolved in water (10 mL) at room temperature and then heated at 60° C. for 24 hours. The reaction was monitored by TLC.

2) After completion of the reaction, the methanol was removed by rotary evaporation, then treated with strong acid cation exchange resin. The filtrate obtained from the filtration of the resin was lyophilized to give a colorless viscous liquid (1.2 g). The crude product was used directly in the next step. MS, m/z: 331.12 [M+H]+

(4) Preparation of cis-[trans-(1R,2R)-diaminocyclohexane] Pt (II) (1-O-D-glucoside-ethane-2-chloro-2,2-dicarboxylate) (I-19)

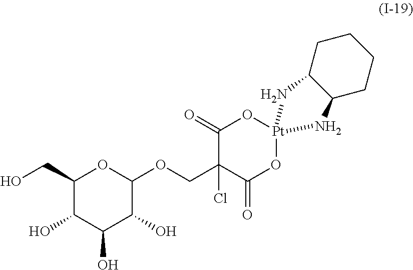

1) 1 g of crude 1-O-D-glucoside-ethane-2-chloro-2,2-dicarboxylic acid was dissolved in water (10 mL), to which was added a freshly prepared aqueous solution of barium hydroxide to adjust the pH to 7, and then stirred at room temperature for 30 minutes.

2) Under the protection of nitrogen, to the reaction solution of 1) was added a solution of trans-(1R,2R)-diaminocyclohexane platinum sulfate (1.3 g) dissolved in water (2 mL) followed by a freshly prepared aqueous solution of barium hydroxide to re-adjust the pH to 7. The reaction mixture was stirred in the dark at a room temperature overnight.

3) After completion of the reaction, the precipitate was removed by centrifugation, the supernatant was collected and separated by semi-preparative HPLC, and the fraction of the collected product was lyophilized by a lyophilizer to obtain the final product (I-19) as a white solid. Yield: 1.0 g.

1H NMR (400 MHz, D20): δ4.87 (d, J=3.6 Hz, 0.8H), 4.43 (d, J=7.2 Hz, 0.2H), 3.00-4.50 (m, 8H), 2.20-2.45 (m, 2H), 1.96 (d, J=12 Hz, 2H), 1.49 (d, J=8 Hz, 2H), 1.12-1.30 (s, 2H), 0.95-1.10 (m, 2H). MS, m/z: 638.16 [M+H]+

### Example 20

### Preparation of diamine Pt (II) (1-O-D-glucoside-ethane-2-chloro-2,2-dicarboxylate) (I-20)

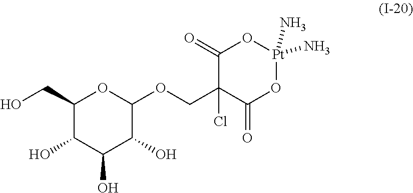

1) 100 mg of 1-O-D-glucoside-ethane-2-chloro-2,2-dicarboxylic acid was dissolved in water (10 mL), to which was added a freshly prepared aqueous solution of barium hydroxide to adjust the pH to 9, and then stirred at room temperature for 30 minutes.

2) Under the protection of nitrogen, to the reaction solution of 1) was added a solution of diamine platinum sulfate (120 mg) dissolved in water (2 mL) followed by a freshly prepared aqueous solution of barium hydroxide to re-adjust the pH to 9. The reaction mixture was stirred in the dark at a room temperature overnight.

3) After completion of the reaction, the precipitate was removed by centrifugation, the supernatant was collected and separated by semi-preparative HPLC, and the fraction of the collected product was lyophilized by a lyophilizer to obtain the final product (I-20) as a white solid. Yield: 97 mg.

1H NMR (400 MHz, D20): δ4.88 (d, J=3.6 Hz, 0.8H), 4.45 (d, J=7.2 Hz, 0.2H), 3.00-4.50 (m, 8H). MS, m/z: 558.13 [M+H]+

### Example 21

### Preparation of bisdiisopropylamine platinum Pt (II) (1-O-D-glucoside-ethane-2-chloro-2,2-dicarboxylate) (I-21)

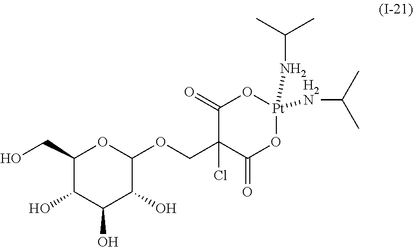

1) 100 mg of 1-O-D-glucoside-ethane-2-chloro-2,2-dicarboxylic acid was dissolved in water (5 mL), to which was added a freshly prepared aqueous solution of barium hydroxide to adjust the pH to 9, and then stirred at room temperature for 30 minutes.

2) Under the protection of nitrogen, to the reaction solution of 1) was added a solution of bisdiisopropylamine platinum platinum sulfate (140 mg) dissolved in water (2 mL) followed by a freshly prepared aqueous solution of barium hydroxide to re-adjust the pH to 9. The reaction mixture was stirred in the dark at a room temperature overnight.

3) After completion of the reaction, the precipitate was removed by centrifugation, the supernatant was collected and separated by semi-preparative HPLC, and the fraction of the collected product was lyophilized by a lyophilizer to obtain the final product (I-21) as a white solid. Yield: 110 mg.

1H NMR (400 MHz, D20): 64.88 (d, J=3.6 Hz, 0.8H), 4.83 (br, 4H), 4.44 (d, J=7.2 Hz, 0.2H), 3.00-4.30 (m, 8H), 2.41 (m, 2H), 1.15-1.30 (m, 12H). MS, m/z: 642.21 [M+H]+

### Example 22

### Preparation of amino isopropylamide platinum (II) (1-O-D-glucoside-propane-3-chloro-3,3-dicarboxylate) (I-22)

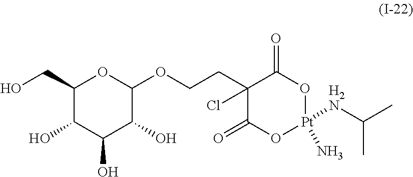

1) 100 mg of crude 1-O-D-glucoside-propane-3-chloro-3,3-dicarboxylic acid was dissolved in water (5 mL), to which was added a freshly prepared aqueous solution of barium hydroxide to adjust the pH to 9, and then stirred at room temperature for 30 minutes.

2) Under the protection of nitrogen, to the reaction solution of 1) was added a solution of amino isopropylamine platinum sulfate (110 mg) dissolved in water (2 mL) followed by a freshly prepared aqueous solution of barium hydroxide to re-adjust the pH to 9. The reaction mixture was stirred in the dark at a room temperature overnight.

3) After completion of the reaction, the precipitate was removed by centrifugation, the supernatant was collected and separated by semi-preparative HPLC, and the fraction of the collected product was lyophilized by a lyophilizer to obtain the final product (I-22) as a white solid. Yield: 110 mg.

1H NMR (400 MHz, D20): δ4.87 (d, J=3.6 Hz, 0.8H), 4.42 (d, J=7.2 Hz, 0.2H), 3.00-4.10 (m, 10H), 2.40-2.45 (m, 1H), 1.15-1.30 (m, 6H). MS, m/z: 614.23 [M+H]+

### Example 23

### Preparation of dimethylamine Pt (II) (1-O-D-glucoside-propane-3-chloro-3,3-dicarboxylate) (I-23)

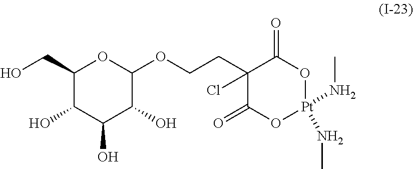

1) 100 mg of crude 1-O-D-glucoside-propane-3-chloro-3,3-dicarboxylic acid was dissolved in water (5 mL), to which was added a freshly prepared aqueous solution of barium hydroxide to adjust the pH to 9, and then stirred at room temperature for 30 minutes.

2) Under the protection of nitrogen, to the reaction solution of 1) was added a solution of dimethylamine platinum sulfate (120 mg) dissolved in water (2 mL) followed by a freshly prepared aqueous solution of barium hydroxide to re-adjust the pH to 9. The reaction mixture was stirred in the dark at a room temperature overnight.

3) After completion of the reaction, the precipitate was removed by centrifugation, the supernatant was collected and separated by semi-preparative HPLC, and the fraction of the collected product was lyophilized by a lyophilizer to obtain the final product (I-23) as a white solid. Yield: 115 mg.

1H NMR (400 MHz, D20): δ4.87 (d, J=3.6 Hz, 0.8H), 4.42 (d, J=7.2 Hz, 0.2H), 3.00-4.10 (m, 10H), 2.47 (s, 6H). MS, m/z: 599.21 [M+H]+

### Example 24

### Preparation of dioctylamine Pt (II) (1-O-D-glucoside-propane-3-chloro-3,3-dicarboxylate) (I-24)

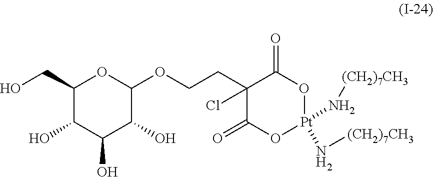

1) 100 mg of crude 1-O-D-glucoside-propane-3-chloro-3,3-dicarboxylic acid was dissolved in water (5 mL), to which was added a freshly prepared aqueous solution of barium hydroxide to adjust the pH to 9, and then stirred at room temperature for 30 minutes.

2) Under the protection of nitrogen, to the reaction solution of 1) was added a solution of dioctylamine platinum sulfate (120 mg) dissolved in water (2 mL) followed by a freshly prepared aqueous solution of barium hydroxide to re-adjust the pH to 9. The reaction mixture was stirred in the dark at a room temperature overnight.

3) After completion of the reaction, the precipitate was removed by centrifugation, the supernatant was collected and separated by semi-preparative HPLC, and the fraction of the collected product was lyophilized by a lyophilizer to obtain the final product (I-24) as a white solid. Yield: 160 mg.

1H NMR (400 MHz, D20): δ4.87 (d, J=3.6 Hz, 0.8H), 4.42 (d, J=7.2 Hz, 0.2H), 3.00-4.15 (m, 10H), 2.40-2.47 (m, 4H), 1.50-1.55 (m, 4H), 1.10-1.40 (m, 20H), 0.85-0.88 (m, 6H). MS, m/z: 796.41 [M+H]+

### Example 25

### Preparation of dicyclopropylamine Pt (II) (1-O-D-glucoside-propane-3-chloro-3,3-dicarboxylate) (I-25)

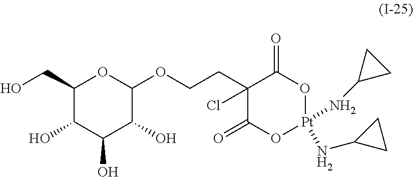

1) 100 mg of crude 1-O-D-glucoside-propane-3-chloro-3,3-dicarboxylic acid was dissolved in water (5 mL), to which was added a freshly prepared aqueous solution of barium hydroxide to adjust the pH to 9, and then stirred at room temperature for 30 minutes.

2) Under the protection of nitrogen, to the reaction solution of 1) was added a solution of dicyclopropylamine platinum sulfate (110 mg) dissolved in water (2 mL) followed by a freshly prepared aqueous solution of barium hydroxide to adjust the pH to 9. The reaction mixture was stirred in the dark at a room temperature overnight.

3) After completion of the reaction, the precipitate was removed by centrifugation, the supernatant was collected and separated by semi-preparative HPLC, and the fraction of the collected product was lyophilized by a lyophilizer to obtain the final product (I-25) as a white solid. Yield: 90 mg.

1H NMR (400 MHz, D20): δ4.87 (d, J=3.6 Hz, 0.8H), 4.83 (br, 4H), 4.42 (d, J=7.2 Hz, 0.2H), 3.00-4.15 (m, 10H), 2.68-2.79 (m, 2H), 0.75-0.95 (m, 8H). MS, m/z: 652.31 [M+H]+

### Experiment-1

To compare the platinum complexes of the present invention with the clinical drugs of cisplatin, carboplatin and oxaliplatin in the aspect of water solubility, the solubility of the representative platinum complexes of the present invention and three clinical drugs in water (1 g) at room temperature has been measured. The solubility of such tested complexes in water and their differences from clinical platinum-based antitumor drugs, namely, cisplatin, carboplatin and oxaliplatin, are presented in Table 2.

TABLE 2

| | Compound | | | | | | | | |
|---|---|---|---|---|---|---|---|---|---|
| | | | | Pt-complex | Pt-complex | Pt-complex | Pt-complex | Pt-complex | Pt-complex |
| | Cisplatin | Carboplatin | Oxaliplatin | of Example 1 | of Example 3 | of Example 4 | of Example 12 | of Example 12 | of Example 20 |
| solubility | 1.0 | 16.6 | 5.5 | 786 | 533 | 423 | 786 | 400 | 900 |
| (mg/1 mL | | | | | | | | | |
| H₂O) | | | | | | | | | |

The above experimental results showed that the water solubility of platinum complexes from the present invention is 400-900 fold higher than the clinical drug cisplatin, and is 70-160 times higher than that of clinical drug oxaliplatin.

Experimental results demonstrated that, in the current invention, through introducing water-soluble sugar substituent to the traditional malonato-platinum complex structure, an unprecedented high water solubility has been achieved.

### Experiment-2

In the following test, antitumor efficacy studies were performed using 8-9 weeks old female CDF1 mice, the average weight of the animal is 20-25 grams. L1210 tumor cells (about 105 cells per mouse) were inoculated intraperitoneally. The water-soluble platinum complexes were used to treat the tumor-bearing animals and the efficacy was compared compared with the clinical platinum antitumor drugs. For the water-soluble platinum complexes of the present invention and carboplatin, 5 wt % mannitol-water solution was used for preparing the corresponding injection, but for cisplatin, 5 wt % mannitol-saline solution was used. Drugs were administered intraperitoneally on day 1 and day 4 after tumor cell transplantation. The number of experimental animals in each group was 6.

The experimental animals were purchased from Vital River Laboratory Animal Technology Co. Ltd., Tumor cells L1210-leukemia cells were purchased from Shanghai An Yan Commercial Trade Co. Ltd.

The increase in life span (ILS) is calculated as follows:

ILS %=[(St/Su)−1]×100%

Wherein, St=the weighted median survival time of treated animals; Su=the weighted median survival time of untreated animals

The results are shown in Table 3:

TABLE 3

| | | Weight | | Survival |
|---|---|---|---|---|
| | Dosage | Changes | | Animals |
| Compound | (mg/Kg) | (g)* | ILS (%) | on Day 42 |
| Untreated Control | / | +1.8 | / | 0/6 |
| Group | | | | |
| Pt-complex of | 50 | +1.1 | >425 | 6/6 |
| Example 1 | 100 | +0 | >425 | 6/6 |
| | 200 | −0.4 | >425 | 6/6 |
| Pt-complex of | 50 | +1.2 | >365 | 6/6 |
| Example 10 | 100 | −0.2 | >365 | 6/6 |
| | 200 | −0.5 | >365 | 6/6 |
| Carboplatin | 24 | +0.6 | 8 | 0/6 |
| | 40 | −0.4 | 22 | 0/6 |
| | 80 | −1.2 | 50 | 1/6 |
| Cisplatin | 1.25 | +1.1 | 20 | 0/6 |
| | 2.5 | −0.6 | 125 | 1/6 |
| | 5.0 | −2.2 | 265 | 3/6 |

*weight changes from the first day to the seventh day

### Experiment-3

The tumor cell proliferation inhibition effect of the water-soluble platinum complexes of the present invention.

The antitumor effect of the invented chlorine-containing water-soluble platinum complexes is achieved by forming intrastrand and interstrand cross-linking DNA alkylating adduct which thereby inhibiting the tumor cell DNA synthesis and replication.

The following experiments are performed to testify the proliferation inhibition effect of the water-soluble platinum complexes of the present invention on different types of human tumor cells.

(1) Test Methods:

Cell Culture Medium:

Containing 10% bovine fetal serum, 1 mM of sodium pyruvate, 2 mM of L-glutamine, 50 U/mL of penicillin, 50 μg/mL of streptomycin

The Main Experimental Apparatus:

MC0-15A Carbon Dioxide Incubator (SANYO, Japan), Inverted Phase Contrast Microscope (Olympus, Japan), Automatic Microplate Reader (U.S. BioTEK ELX808), Low Temperature Refrigerator (MDF-V5410, Japan), Clean Bench (Suzhou Medical Apparatus Factory), Micropipettes (GILSON, France), Automatical Pure Water Distillatory (1810B, Shanghai).

Reagents:

MTS: CellTiter96 Aqueous MTS Reagent Powder, Promega

PMS: Phenazine methosulfate (PMS), Sigma-Aldrich

DPBS: Sigma-Aldrich

Tumor Cells:

Human tumor cells: dul45-human prostate cancer; MCF-7-human breast cancer; SKOV3-human ovarian carcinoma, HT-29-human colon cancer; A549-human non-small cell lung cancer (adenocarcinoma), H460-human non-small cell lung cancer (large cell carcinoma), and animal tumor cells: L1210-mice leukemia cells used in the following activity test experiments were all purchased from Shanghai An Yan Commercial Trade Co., Ltd.

Cytotoxicity Test:

MTS test method was used in cytotoxicity assay. The tumor cells of logarithmic phase were collected, and then the concentration of cell suspension was adjusted, 100 μL of the cell suspension was added to each well, the cells were placed at 1000-10000 cells/well (edge well filled with sterile PBS). Cells were incubated at 37° C. with 5% CO2 to make cell monolayer overspread the bottom of each well (96-well flat-bottomed microplate). 100 μL of different concentrations of the test compounds was added to each well. Each condition was measured in five replicates. The microplate was incubated at 37° C. with 5% CO2 for 96 h and checked with inverted microscope. MTS working reagent: To 2 mL of MTS (2 mg/mL, prepared by DPBS) was added 100 μL of PMS (1 mg/mL, prepared by DPBS). The cell culture medium was discarded after centrifugation, the cell culture plate was carefully washed 2-3 times with PBS. Before detecting the absorbance value (OD), to each sample containing well was added 100 μL of cell culture medium, then 20 μL of MTS working reagent was added. After incubation at 37° C. with 5% CO2 for 2 h, the OD (optical density) value was detected at 490 nm

Control group: the conditions are the same as the above without adding the active ingredient of antitumor agents, and the OD value was detected at 490 nm on the end of the experiment.

Cyototoxicity IC50:

The cell inhibiting rate of the drugs to tumor cell growth was calculated according to the following formula:

1) Cell viability (%)=OD of treated group/OD of control group×100%

2) The cell viabilities under the different drug concentrations were determined, and then plotted against drug concentration. The IC50 value is the corresponding concentration in the obtained curve when the cell viability was 50%.

Each drug concentration was repeated for five times, the cell viability was determined by taking the average of the OD values.

(2) Experimental Results:

Symbols in figures representing the names of tumor cells are as follows: dul45-human prostate cancer; MCF-7-human breast cancer; SKOV3-human ovarian carcinoma; HT-29-human colon cancer; A549-human non-small cell lung cancer (adenocarcinoma); H460-human non-small cell lung cancer (large cell carcinoma)

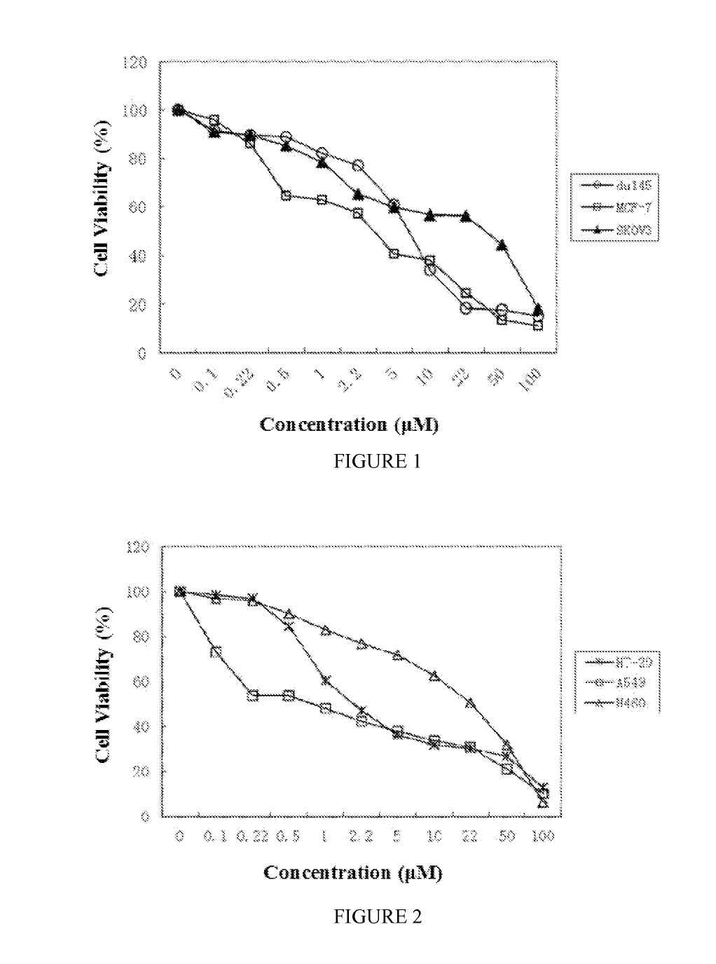

FIG. 1 and FIG. 2 show the cytotoxicity of the platinum complex prepared in Example 1;

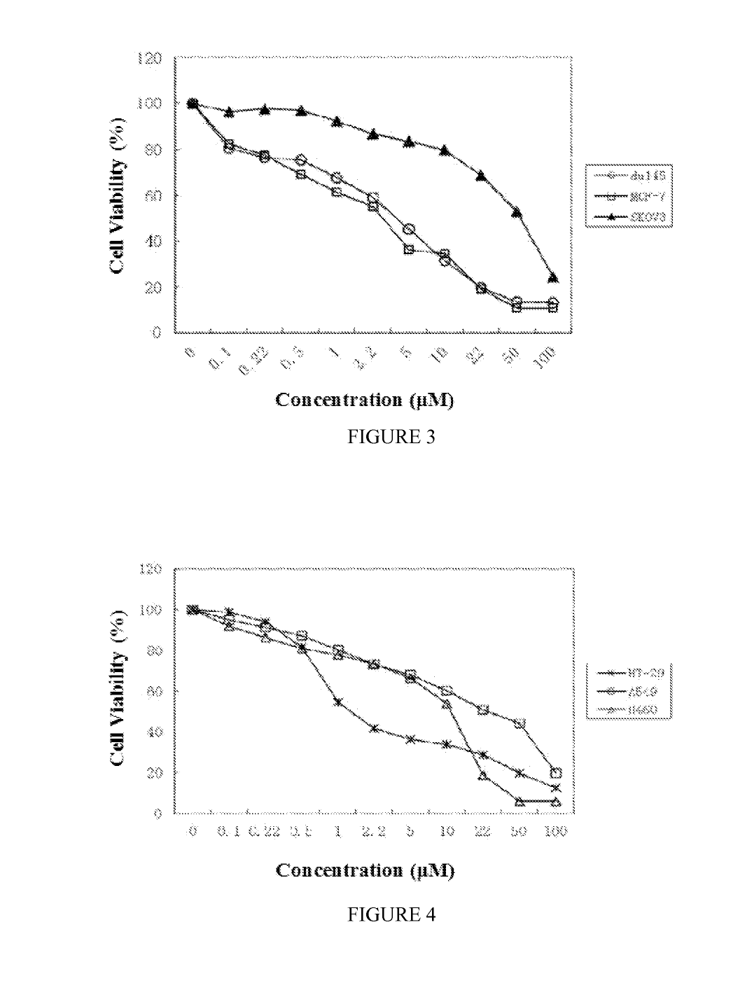

FIG. 3 and FIG. 4 show the cytotoxicity of the platinum complex prepared in Example 5;

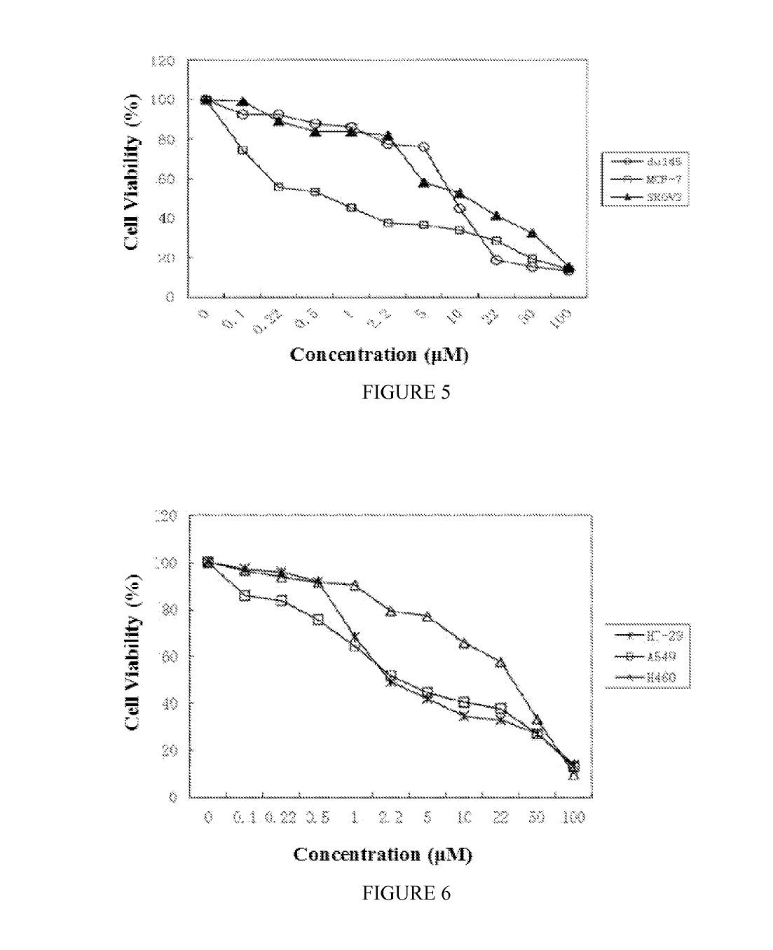

FIG. 5 and FIG. 6 show the cytotoxicity of the platinum complex prepared in Example 9;

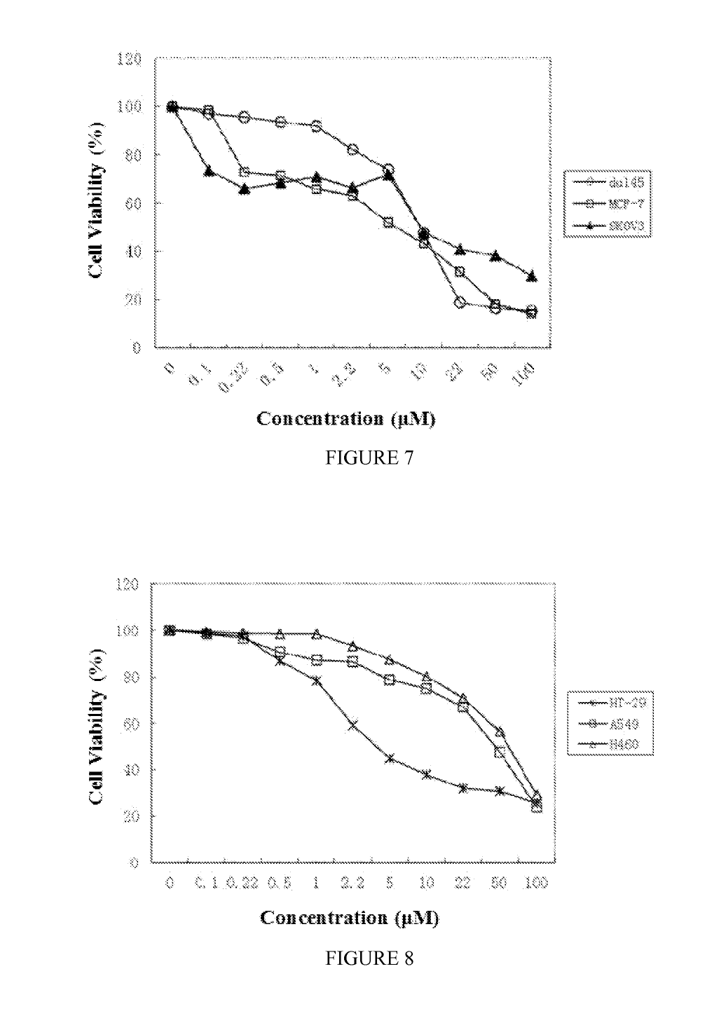

FIG. 7 and FIG. 8 show the cytotoxicity of the platinum complex prepared in Example 10.

In order to show the efficacy trend of the complexes more clearly, standard error bar in all curves in the graph is omitted.

### Experiment-4

Anti-tumor effect of the water-soluble platinum complexes of the present invention on animal xenograft models

(1) Test methods: anti-tumor effect studies were performed using 5-6 weeks old Nu/nu male nude mice which were purchased from Beijing Vital River Laboratory Animal Technology Co. Ltd. Experimental animals were kept under the SPF level environment in the IVC systems. All animals had a free access to the food and water, the room temperature was 20 to 25° C., the humidity was 40% to 70%, and the alternation of day and night was 12 h/12 h.

Colorectal cancer DLD-1 cells were collected and subcutaneously injected into the armpit of each nude mouse, and then the model of tumor bearing mice was established. When the tumor volume grew to 150˜300 cm3, according to the tumor volume and weight, the mice were equally divided into 5 groups (saline group, Example 6 group, Example 18 group, Example 25 group, oxaliplatin group, 10 animals in each group). Experimental compounds were injected intraperitoneally once a week, and the volume of administration is 10 mL/kg body weight. After four weeks of the drug treatment, the mice were continually fed with a normal diet, the tumor growth and the anti-tumor efficacy of the tested compounds were dynamically observed by measuring tumor volume and size on alternate days. Experimental observation was continued for 61 days after grouping.

The calculation formula of tumor volume: V=½×a×b2. Wherein, a and b are the tumor length and width, the tumor volume was calculated based on the measurements. Percent tumor volume increase (%)=(Vt−V0)/V0×100. V0 is the tumor volume before administration (that is d0); Vt is the tumor volume after administration.

(2) Administration dosage: according to the pre-measured maximum tolerated dose (MTD) of the drugs on the same nude mice, 70% of the MTD was used as the administration dosage. Wherein clinical oxaliplatin was 7.5 mg/kg body weight, the platinum complex of Example 6 was 45 mg/kg body weight, the platinum complex of Example 18 was 28 mg/kg body weight, and the platinum complex of Example 25 was 20 mg/kg body weight. Drugs were dissolved in sterile distilled water using ultrasound before injection.

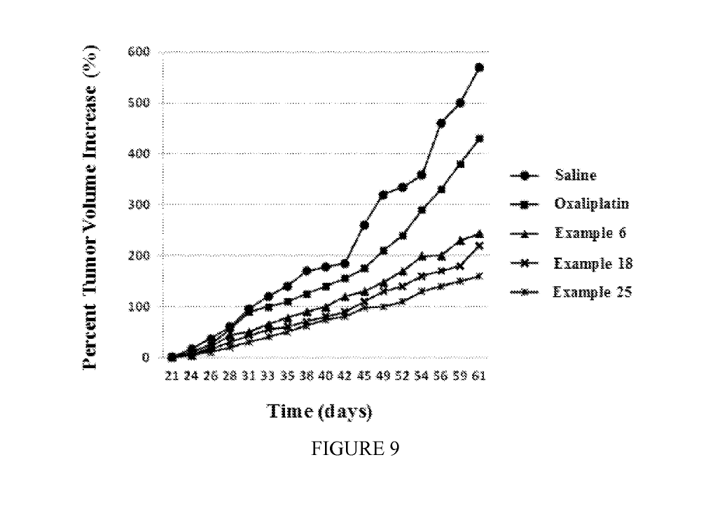

(3) Experimental results: the experimental results show that the water-soluble platinum complexes of the present invention have a significant advantage over clinical drug oxaliplatin on tumor inhibition effect, especially in aspect of long term suppression effect on tumor growth after drug treatment being stopped. This result implicates that, in spite of the extremely high water solubility (theoretically can not cross over the cell membrane), the invented platinum complexes have selected accumulation in tumor cells and tissues and therefore, exhibited improved tumor targeting effect (See FIG. 9). In order to clearly show the efficacy trend of the complexes, standard error bar in all curves in the graph is omitted.

The platinum complexes of the present invention can be used to prepare medicines for cancer prevention and treatment. These medicines were usually prepared by using an effective amount of one or several platinum complexes of the present invention together with the pharmaceutically acceptable vehicles or diluents. These pharmaceutically acceptable excipients such as starch, glucose, dextrin, fructose, maltose, lactose, gelatin, sucrose, hydroxyl cellulose, hydroxypropyl methyl cellulose, silicon dioxide, stearic acid, sodium starch glycolate, water, ethanol, sodium chloride and the like, were selected according to different needs of dosage form. In addition, according to the requirements of pharmaceutical preparation, these excipients may also include small amounts of pH buffering agents, stabilizing agents, etc.

Experiments show that the platinum complexes of the present invention have a good anti-tumor activity. The chlorine-containing water-soluble platinum complexes of the present invention are superior to the widely used clinical drugs: cisplatin, carboplatin or oxaliplatin in antitumor efficacy as tested in xenograft models and different tumor cells such as colon cancer, breast cancer, prostate cancer, lung cancer, etc. Furthermore, in the strong cisplatin-resistant Leukemia-L1210 tumor cells, the invented water soluble platinum complexes inhibited superior antitumor efficacy compared with cisplatin.

Because the water solubility of the invented platinum complexes have been increased by tens or hundreds of times as compared to the clinical platinum antitumor drugs, thus the drug excretion in the kidney can be improved and therefore, the high renal toxicity caused by the traditional platinum antitumor drugs can be reduced. Furthermore, the feature of the high water solubility makes the drugs easy to prepare and formulated for clinically use.

Due to the extremely high water solubility, the administration route of the complexes of the present invention is not restrictive. The dose depends not only on the age of the subject, the weight of the subject and the condition of the subject, but also on the type of tumor, the nature of tumor and the severity of tumor. Generally, for an adult subject, the dose was preferably used in an amount of 10 mg to 1 g per day, usually once or several times every one to three weeks.

1-10. (canceled)

11. A water-soluble platinum complex for tumor treatment, of the formula (I): 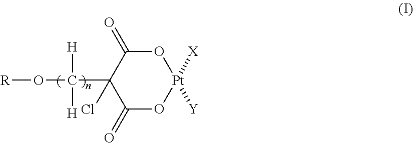
Wherein:
X and Y are ligand. X and Y are same or independently chosen from NH3, C1-C8 aliphatic primary amines, C3-C8 cyclic primary amines or X and Y together form a 1,2-cyclohaxanediamine chosen from trans-(1R,2R)-cyclohexanediamine, trans-(1S,2S)-cyclohexanediamine, cis-(1R,2S)-cyclohexanediamine, cis-(1S-2R)-cyclohexanediamine, trans-meso-1,2-cyclohexanediamine, cis-meso-1,2-cyclohexanediamine;
n is 2 or 3;
R is chosen from

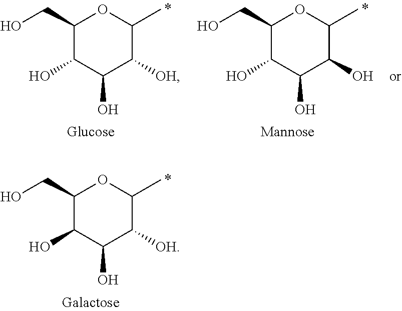

12. An intermediate of formula (III) for preparing the complex of claim 11: 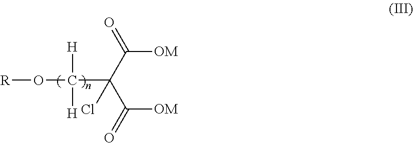
Wherein:
Each M chosen from hydrogen or metals in Group IA of the elemental periodic table; or M together represents a transition metal in Group IIA of the elemental periodic table;
n is 2 or 3;
R is chosen from

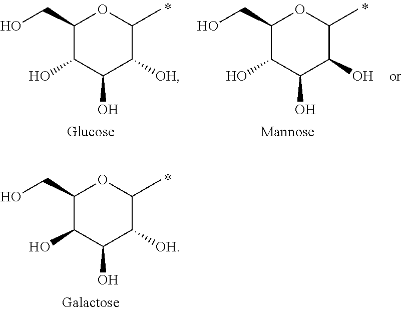

13. A method of preparing the complex (I) of claim 11, which includes the following step:
Reacting compound (II) with a water solution of intermediate (III) at a pH range of 7-9, or reacting compound (II) with intermediate (III) in water in the present of inorganic base, where the compound (II) is: 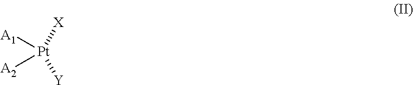
Wherein:
X and Y are ligand. X and Y are same or independently chosen from NH3, C1-C8 aliphatic primary amines, C3-C8 cyclic primary amines or X and Y together form a 1,2-cyclohaxanediamine chosen from trans-(1R,2R)-cyclohexanediamine, trans-(1S,2S)-cyclohexanediamine, cis-(1R,2S)-cyclohexanediamine, cis-(1S-2R)-cyclohexanediamine, trans-meso-1,2-cyclohexanediamine, cis-meso-1,2-cyclohexanediamine;
A1 and A2 are same or independently chosen from —OH, —NO₃, —C104, or A1 and A2 together represents —SO4, —CO3;
The formula of intermediate (III) is: 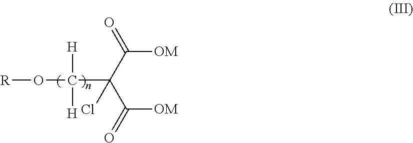
Wherein:
Each M chosen from hydrogen or metals in Group IA of the elemental periodic table; or M together represents a transition metal in Group IIA of the elemental periodic table;
n is 2 or 3;
R is chosen from

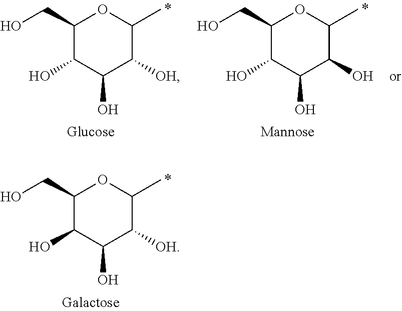

14. The method of claim 13, wherein each M is Hydrogen, Sodium or M together represents Barium;

15. The method of claim 13, wherein the inorganic base is NaOH, KOH, LiOH, Ba(OH)2 or Cs(OH)2.
